(19) United States
(12) Patent Application Publication
(10) Pub. No.: US 2022/0079143 A1
(43) Pub. Date: Mar. 17, 2022

(54) OIL-IN-WATER TYPE PEST REPELLENT COMPOSITION

(71) Applicant: Kao Corporation, Chuo-ku, Tokyo (JP)

(72) Inventor: Hiroyuki TAKIZAWA, Lumpini, Bangkok (TH)

(73) Assignee: Kao Corporation, Chuo-ku, Tokyo (JP)

(21) Appl. No.: 17/417,160

(22) PCT Filed: Dec. 19, 2019
(86) PCT No.: PCT/JP2019/049738

§ 371 Date: Jun. 22, 2021

(30) Foreign Application Priority Data
Dec. 28, 2018 (JP) 2018-248692

(51) Int. Cl. A01N 25/04; A61K 8/895; A01N 61/02; A61K 8/06; A61Q 17/02; A61K 8/92

(52) U.S. Cl. CPC A01N 25/04; A61K 8/895; A61K 8/92; A61K 8/062; A61Q 17/02; A01N 61/02

(57) ABSTRACT

The present invention relates to an oil-in-water type pest repellent composition containing the following components (A) to (C):
(A) 13% by mass or more and 50% by mass or less of at least one non-volatile liquid oily component selected from the group consisting of a silicone oil, an ester oil, an ether oil, a hydrocarbon oil, an aliphatic alcohol, and a polyhydric alcohol, the non-volatile liquid oily component having a surface tension at 25° C. of 40 mN/m or less and a viscosity at 23° C. as measured with a B-type rotational viscometer of 400 mPa·s or less;
(B) 0.5% by mass or more and 35% by mass or less of hydrophobic particles having a volume median particle diameter of 0.1 μm or more and 40 μm or less; and
(C) 30% by mass or more and 86.5% by mass or less of water.

### FIELD OF THE INVENTION

The present invention relates to an oil-in-water type pest repellent composition.

### BACKGROUND OF THE INVENTION

Pests, for example, flying pests, such as mosquitoes and flies, carry pathogens to animals, such as humans and are factors that cause infectious diseases and dermatitis. In particular, some mosquitoes are hygienically very harmful insects because they carry pathogens, such as dengue fever, Zika fever, yellow fever, encephalitis, and malaria.

Conventionally, in order to protect themselves from such flying pests, a method of spraying an insecticide or applying a repellent onto the skin surface has been widely used. As a typical repellent, DEET (N,N-diethyl-toluamide) is commonly used.

Pests, such as mosquitoes, possess chemoreceptive systems, such as thermal receptors that sense the body temperature of animals, olfactory receptors that sense volatile substances, such as body odors, and carbon dioxide receptors that sense carbon dioxide, and detect the animals by them. However, DEET repels pests by modulating the chemoreceptive system of such pests and neutralizing the cognitive sensation of the pest.

However, DEET involves such problems that it has an unpleasant odor, and it is also required that a fixed amount or more of the compound is blended to exhibit a sufficient sustaining repellent effect. Furthermore, it is known that DEET causes allergy or chopped skin depending upon a human, and the use amount or the number of uses for infants and people with sensitive skin is limited.

Then, utilization of natural essential oils as a repellent component is studied. Natural essential oils, such as citronella oil, lemon eucalyptus oil, lemongrass oil, orange oil, and cassia oil, are also used for candles and aroma lotions and high in safety for a human body. However, their repellent effect against pests is not sufficient, and their practicality is problematic.

Besides, various proposals have been made for pest repellents and pest repellent compositions.

For example, PTL 1 describes that a skin protective agent having a fluorine-polyether co-modified silicone and a UV protective component blended therein is blended with an insect repellent, such as DEET.

PTL 2 discloses a pest repellent containing carane-3,4-diol and a silicone oil having a viscosity of 5,000 cSt or less at 25° C. In addition, PTL 3 discloses an insecticide containing a silicone-based compound as an active ingredient.

### CITATION LIST

### Patent Literature

PTL 1: JP 8-59447 A
PTL 2: JP 8-81307 A
PTL 3: JP 2006-36759 A

### SUMMARY OF THE INVENTION

The present invention provides the following [1] to [7].

[1] An oil-in-water type pest repellent composition or pest stay inhibition composition containing the following components (A) to (C):

(A) 13% by mass or more and 50% by mass or less of at least one non-volatile liquid oily component selected from the group consisting of a silicone oil, an ester oil, an ether oil, a hydrocarbon oil, an aliphatic alcohol, and a polyhydric alcohol, the non-volatile liquid oily component having a surface tension at 25° C. of 40 mN/m or less and a viscosity at 23° C. as measured with a B-type rotational viscometer of 400 mPa·s or less;

(B) 0.5% by mass or more and 35% by mass or less of hydrophobic particles having a volume median particle diameter of 0.1 μm or more and 40 μm or less; and

(C) 30% by mass or more and 86.5% by mass or less of water.

[2] An oil-in-water type mosquito repellent composition or mosquito stay inhibition composition containing the following components (A) to (C) and having the content of other pest repellent than the component (A) of 15% by mass or less:

(A) 13% by mass or more and 50% by mass or less of at least one non-volatile liquid oily component selected from the group consisting of a silicone oil, an ester oil, an ether oil, a hydrocarbon oil, an aliphatic alcohol, and a polyhydric alcohol, the non-volatile liquid oily component having a surface tension at 25° C. of 40 mN/m or less and a viscosity at 23° C. as measured with a B-type rotational viscometer of 400 mPa·s or less;

(B) 0.5% by mass or more and 35% by mass or less of hydrophobic particles containing resin particles constituted of at least one resin selected from the group consisting of a silicone resin, an acrylic resin, and a polyurethane resin and having a volume median particle diameter of 0.1 μm or more and 40 μm or less; and

(C) 30% by mass or more and 86.5% by mass or less of water. [3] An oil-in-water type mosquito repellent composition or mosquito stay inhibition composition containing the following components (A) to (C), with a mass ratio [(A)/(B)] of the component (A) to the composition (B) being 2 or more and 5 or less, and having the content of other pest repellent than the component (A) of 1% by mass or less:

(A) 13% by mass or more and 50% by mass or less of at least one non-volatile liquid oily component selected from the group consisting of a silicone oil, an ester oil, an ether oil, a hydrocarbon oil, an aliphatic alcohol, and a polyhydric alcohol, the non-volatile liquid oily component having a surface tension at 25° C. of 40 mN/m or less and a viscosity at 23° C. as measured with a B-type rotational viscometer of 400 mPa·s or less;

(B) 0.5% by mass or more and 35% by mass or less of hydrophobic particles having a volume median particle diameter of 1.5 μm or more and 25 μm or less; and

(C) 30% by mass or more and 86.5% by mass or less of water.

[4] An oil-in-water type mosquito repellent composition or mosquito stay inhibition composition containing the following components (A) to (D), with a mass ratio [(A)/(B)] of the component (A) to the composition (B) being 2 or more and 5 or less and a mass ratio [(A)/(C)] of the component (A) to the component (C) being 0.2 or more and 1.2 or less, and having the content of other pest repellent than the component (A) of 1% by mass or less:

(A) 13% by mass or more and 50% by mass or less of at least one non-volatile liquid oily component selected from the group consisting of a silicone oil, an ester oil, and a hydrocarbon oil, the non-volatile liquid oily component having a surface tension at 25° C. of 40 mN/m or less and a viscosity at 23° C. as measured with a B-type rotational viscometer of 400 mPa·s or less;

(B) 0.5% by mass or more and 35% by mass or less of hydrophobic particles having a volume median particle diameter of 1.5 μm or more and 25 μm or less;

(C) 30% by mass or more and 86.5% by mass or less of water; and

(D) 0.05% by mass or more and 3% by mass or less of a thickener.

[5] The oil-in-water type mosquito repellent composition or mosquito stay inhibition composition as set forth in [3] or [4], wherein the component (B) contains resin particles constituted of at least one resin selected from the group consisting of a silicone resin, an acrylic resin, and a polyurethane resin.
[6] A method for repelling pests, including applying the composition as set forth in any one of the [1] to [5] onto the skin surface of a human.
[7] A method for inhibiting pest stay, including attaching the composition as set forth in any one of the [1] to [5] onto limbs of a pest to prevent the pest from staying on the skin of a human.

### DETAILED DESCRIPTION OF THE INVENTION

### [Pest Repellent Composition and Pest Stay Inhibition Composition]

The pest repellent composition and the pest stay inhibition composition of the present invention (these will be hereinafter also collectively referred to as “composition of the present invention”) are each an oil-in-water type composition containing the following components (A) to (C):

(A) 13% by mass or more and 50% by mass or less of at least one non-volatile liquid oily component selected from the group consisting of a silicone oil, an ester oil, an ether oil, a hydrocarbon oil, an aliphatic alcohol, and a polyhydric alcohol, the non-volatile liquid oily component having a surface tension at 25° C. of 40 mN/m or less and a viscosity at 23° C. as measured with a B-type rotational viscometer of 400 mPa·s or less;

(B) 0.5% by mass or more and 35% by mass or less of hydrophobic particles having a volume median particle diameter of 0.1 μm or more and 40 μm or less; and

(C) 30% by mass or more and 86.5% by mass or less of water.

The composition of the present invention is an oil-in-water type composition containing specified amounts of the components (A) to (C), which is excellent in repellent sustaining effect against pests, particularly flying pests and is safe, and further, is good in feeling on the occasion of applying onto the skin and excellent in long-term storage stability.

Of the pest repellents, those to be applied to the skin are desired to be provided with performances such that they are excellent in not only initial pest repellent effect but also sustainability of the repellent effect and are safe for a human body, and have a good feeling of use on the occasion of applying onto the skin and storage stability. However, as mentioned above, it is the present situation that currently usable major pest repellents do not thoroughly meet these desires.

For example, it is known that existing pest repellents, such as DEET, as described in PTL 1 and PTL 2, have an unpleasant odor and cause allergy or chopped skin depending upon a human, and the use amount for infants is limited depending upon a country. Under these circumstances, there is demanded a repellent having higher safety for the human body and capable of being safely used by infants and people with sensitive skin.

In addition, different from the insecticide described in PTL 3, a repellent composition of a type which is applied onto the human skin and used is desired to be provided with performances such that it is excellent in not only initial pest repellent effect but also sustainability of the repellent effect and has a good feeling on the occasion of applying onto the skin and storage stability.

However, in the existing circumstances, a composition in which a pest repellent effect and sustainability thereof, a good feeling on the occasion of applying onto the skin, and storage stability are made compatible with each has not been found yet.

A problem of the present invention is to provide an oil-in-water type pest repellent composition which is excellent in the pest repellent sustaining effect against pests, particularly flying pests, is safe, is good in the feeling on the occasion of applying onto the skin, and is excellent in the long-term storage stability.

The present inventor made extensive and intensive investigations. As a result, it has been found that an oil-in-water type pest repellent composition containing prescribed amounts of a non-volatile liquid oily component having specified surface tension and viscosity, hydrophobic particles having a specified volume median particle diameter, and water is able to solve the aforementioned problem.

In accordance with the present invention, it is possible to provide an oil-in-water type pest repellent composition and an oil-in-water type pest stay inhibition composition, each of which is excellent in the pest repellent sustaining effect against pests, particularly flying pests, is safe, is good in the feeling on the occasion of applying onto the skin, and is excellent in the long-term storage stability, a method for repelling pests, and a method for inhibiting pest stay.

In the present invention, the term “repellent effect against pests” means that even if the pest comes into contact with a target, it does not stay on that place but leaves away immediately, and the composition of the present invention is corresponding to a contact repellent composition. That is, the pest repellent effect of the present invention is different from a repellent or an insect repellent that repels the pest such that a pest does not contact with the target, such as one that does not attract the pest to the target and one making the pest not approach the target, and an insecticide having an insecticidal activity against the pest and exterminating the pest.

The composition of the present invention exerts an effect of repelling pests in such a manner that even if a pest, such as a mosquito, lands on the target having the composition of the present invention applied or attached thereonto, the pest does not stay on that place but leaves away immediately. Specifically, the composition of the present invention has an effect of preventing the pest, such as a mosquito, from staying in a predetermined area on the skin surface of an animal, such a human, after landing thereon, for a time enough to pierce the animal, for example, 1 second or more. Such an effect is based on an unprecedented pest repellent principle, and is safe without side effects, such as chopped skin. In consequence, the composition of the present invention can be used as a pest stay inhibition composition.

In the present invention, the term “repellent sustaining effect against pests” means that after applying the composition of the present invention to the target, even after a lapse of time, the aforementioned repellent effect is sustained. In the following description, in the present invention, the repellent sustaining effect particularly against flying pests is expressed simply as “repellent sustaining effect”.

The composition of the present invention contains, as the component (A), at least one non-volatile liquid oily component selected from the group consisting of (a) a silicone oil, (b) an ester oil, (c) an ether oil, (d) a hydrocarbon oil, (e) an aliphatic alcohol, and (f) a polyhydric alcohol, the non-volatile liquid oily component having a surface tension at 25° C. of 40 mN/m or less and a viscosity at 23° C. as measured with a B-type rotational viscometer of 400 mPa·s or less. In view of the fact that the composition of the present invention is excellent in the repellent sustaining effect against pests, particularly flying pests by containing the component (A). Although the reason for this is not elucidated yet, the following may be considered.

The present inventor and others have found that a flying pest, such as a mosquito, has properties of avoiding stay on the surface where the limb gets wet in order to avoid the attractive force generated from the landing surface when the limb becomes wet. Since the limb of a flying pest, such as a mosquito, is hydrophobic, a liquid oily component having a surface tension at 25° C. of 40 mN/m or less has a high affinity with the limb of the flying pest. In addition, in a liquid oily component having a viscosity of 400 mPa·s or less, when the limb of the flying pest comes into contact with the liquid oily component, the contact area between the liquid oily component and the limb of the flying pest becomes sufficiently large within a short time. Therefore, it may be considered that when the flying pest lands on the surface onto which the component (A) is applied or attached, the limb gets wet, and at that time, the flying pest avoids the attractive force generated from the landing surface and jumps off without staying at the landing point.

The landing in the present invention refers to the contact of the flying pest, such as a mosquito, with the target for less than 1 second. In addition, the stay in the present invention refers to the matter that the flying pest, such as a mosquito, is kept in contact with the target for 1 second or more.

In view of the fact that the composition of the present invention further contains the component (B) in addition to the component (A), the repellent sustaining effect due to the component (A) is improved, and further, when the composition is applied onto the skin, the feeling becomes good.

### <Component (A): Non-Volatile Liquid Oily Component>

The component (A) which the composition of the present invention contains is at least one non-volatile liquid oily component selected from the group consisting of (a) a silicone oil, (b) an ester oil, (c) an ether oil, (d) a hydrocarbon oil, (e) an aliphatic alcohol, and (f) a polyhydric alcohol, the non-volatile liquid oily component having a surface tension at 25° C. of 40 mN/m or less and a viscosity at 23° C. as measured with a B-type rotational viscometer of 400 mPa·s or less.

The component (A) is preferably liquid at 20° C., more preferably liquid at 15° C., and still more preferably liquid at 10° C. from the viewpoint of facilitating application to the skin and improving the repellent sustaining effect.

The term “liquid” of the liquid oily component means one which is determined to be a liquid in a liquid-solid determination test according to the American Society for Testing and Materials Standards “ASTM D4359-90: Standard Test Method for Determining Whether a Material is a Liquid or Solid”.

The component (A) is preferably a sparingly water-soluble or water-insoluble component from the viewpoint of improving the repellent sustaining effect. Specifically, the dissolution amount in 100 g of water at 20° C. is preferably 1 g or less, more preferably 0.5 g or less, and still more preferably 0.1 g or less, and still more preferably, it is substantially 0 g.

The liquid oily component of the component (A) which is used in the present invention is a non-volatile liquid oily component from the viewpoint of improving the repellent sustaining effect. Here, the non-volatile liquid oily component in the present invention is one in which a volatilization rate after drying at 25° C. for 60 minutes at 1 atm is 50% or less, and preferably one in which a volatilization rate after drying at 25° C. for 120 minutes at 1 atm is 50% or less. The evaluation of volatilization rate is measured according to the German test standard DIN 53249, and specifically, it is performed by the following procedures 1 to 4.

1. The weight (P (g)) of a round filter paper having a diameter of 150 mm is measured.

2. A sample of 0.3 g of the component (A) is dropped on the round filter paper with a pipette, and immediately thereafter, the weight (W₀ (g)) of the filter paper is measured.

3. The weight of the filter paper is measured by weighing with accuracy of about 0.001 g at 25° C. in 5 minute intervals in the absence of aeration. After measuring 2, the weight of the filter paper measured after a lapse of 60 minutes is designated as W₆₀ (g).

4. A value derived from the calculation equation [{((W₀−P)−(W₆₀−P))/(W₀−P)}×100] is designated as the volatilization rate (%).

As the volatilization rate is lower, on the occasion of applying onto the skin, the liquid oily component remains for a longer time, and therefore, the repellent sustaining effect becomes higher.

In the case of using two or more liquid oily components, the volatilization rate of the liquid oily component means a volatilization rate as a mixture of the two or more liquid oily components. In consequence, so long as the volatilization rate of the mixture falls within the aforementioned range, liquid oily components having a volatilization rate of more than 50% after drying at 25° C. for 60 minutes at 1 atm may be combined and used.

The surface tension at 25° C. of the component (A) is preferably 15 mN/m or more, and more preferably 17 mN/m or more from the viewpoint of availability, and it is 40 mN/m or less, preferably 30 mN/m or less, more preferably 28 mN/m or less, still more preferably 25 mN/m or less, yet still more preferably 23 mN/m or less, and even yet still more preferably 21 mN/m or less from the viewpoint of improving the repellent sustaining effect. In addition, even a material having a surface tension at 25° C. of more than 40 mN/m by itself develops the repellent sustaining effect so long as it is mixed with other liquid oily component to lower the surface tension.

The viscosity at 23° C. of the component (A) as measured with a B-type rotational viscometer is preferably 1 mPa·s or more from the viewpoint of suppressing volatility and improving the repellent sustaining effect, and it is 400 mPa·s or less, preferably 300 mPa·s or less, more preferably 210 mPa·s or less, still more preferably 100 mPa·s or less, yet still more preferably 60 mPa·s or less, even yet still more preferably 40 mPa·s or less, and even still more preferably 30 mPa·s or less from the viewpoint of improving the repellent sustaining effect and the viewpoint of securing the long-term storage stability. In the case of using two or more liquid oily components having a different viscosity from each other, the viscosity as a mixture of these liquid oily components is meant.

The surface tension and the viscosity of the component (A) are measured by the methods described in the section of Examples.

### (a) Silicone Oil

As the silicone oil, at least one selected from the group consisting of dimethylpolysiloxane, dimethiconol (dimethylpolysiloxane having a hydroxy group in a terminal thereof), methylphenylpolysiloxane, and modified silicone is preferred from the viewpoint of improving the repellent sustaining effect.

Examples of the modified silicone include amino-modified silicone (dimethylpolysiloxane having an amino group), polyether-modified silicone, glyceryl-modified silicone, amino derivative silicone, carboxy-modified silicone, fatty acid-modified silicone, alcohol-modified silicone, aliphatic alcohol-modified silicone, epoxy-modified silicone, fluorine-modified silicone, and alkyl-modified silicone.

Of the silicone oils, at least one selected from the group consisting of dimethylpolysiloxane, dimethiconol, methylphenylpolysiloxane, and modified silicone is preferred, at least one selected from the group consisting of dimethylpolysiloxane, dimethiconol, and polyether-modified silicone is more preferred, and dimethylpolysiloxane is still more preferred from the viewpoint of improving the repellent sustaining effect and the viewpoint of facilitating a feeling on the occasion of applying onto the skin.

Examples of the dimethylpolysiloxane include at least one selected from the group consisting of a linear dimethylpolysiloxane and a cyclic dimethylpolysiloxane. Of these, a linear dimethylpolysiloxane is more preferred from the viewpoint of improving the repellent sustaining effect.

Examples of a commercially available product of the linear dimethylpolysiloxane include KF-96 Series, manufactured by Shin-Etsu Chemical Co., Ltd.; SH200C Series, 2-1184 Fluid, manufactured by Dow Corning Toray Co., Ltd.; and Silsoft DML, Element 14 PDMS 5-JC, Element 14 PDMS 10-JC, and Element 14 PDMS 20-JC, all of which are manufactured by Momentive Performance Materials, Inc.

### (b) Ester Oil

As the ester oil, any of ester oils represented by the following general formulae (1) to (3) and a dialkyl carbonate compound represented by the following general formula (4) are preferred from the viewpoint of improving the repellent sustaining effect.

R¹—COO—R² (1)

In the general formula (1), R¹ represents a linear or branched alkyl group having 7 or more and 23 or less carbon atoms, or an aromatic hydrocarbon group having 6 or more and 24 or less carbon atoms, which may be substituted with a hydroxy group; and R² represents a linear or branched alkyl group or alkenyl group having 1 or more and 22 or less carbon atoms.

In the case where R¹ is an alkyl group, the carbon number thereof is preferably 7 or more, and more preferably 9 or more from the viewpoint of improving the repellent sustaining effect, and it is preferably 21 or less, and more preferably 17 or less from the viewpoint of facilitating a feeling on the occasion of applying onto the skin. In addition, in the case where R¹ is an aromatic hydrocarbon group, the carbon number thereof is preferably 8 or more, and more preferably 10 or more from the viewpoint of improving the repellent sustaining effect, and it is preferably 22 or less, and more preferably 20 or less from the viewpoint of facilitating a feeling on the occasion of applying onto the skin.

R² is a linear or branched alkyl group or alkenyl group having preferably 20 or less carbon atoms, and more preferably 18 or less carbon atoms from the viewpoint of facilitating a feeling on the occasion of applying onto the skin. In addition, at least one of R¹ and R² is preferably a branched alkyl group from the viewpoint of facilitating a feeling on the occasion of applying onto the skin.

Examples of the ester oil represented by the general formula (1) is at least one selected from the group consisting of myristyl 2-ethylhexanoate, cetyl 2-ethylhexanoate, stearyl 2-ethylhexanoate, isodecyl octanoate, isocetyl octanoate, isononyl isononanoate, isotridecyl isononanoate, cetearyl isononanoate, octyl propylheptanoate, methyl laurate, isopropyl myristate, octyldodecyl myristate, isopropyl palmitate, 2-ethylhexyl palmitate, isocetyl palmitate, 2-ethylhexyl stearate, isocetyl stearate, isotridecyl stearate, isopropyl isostearate, octyl isostearate, isocetyl isostearate, isostearyl isostearate, 2-ethylhexyl hydroxystearate, methyl oleate, oleyl oleate, isobutyl oleate, oleyl erucate, an alkyl benzoate (carbon number of alkyl: 12 to 15), and diethylhexyl naphthalenedicarboxylate.

(R³O)—CH₂CH(OR⁴)—CH₂(OR⁵) (2)

In the general formula (2), R³, R⁴, and R⁵ are each independently a hydrogen atom or a group represented by the following general formula (2-1), provided that all of them are not a hydrogen atom at the same time.

—CO—R⁶ (2-1)

In the formula, R⁶ represents an alkyl group or alkenyl group having 7 or more and 23 or less carbon atoms, and preferably 17 or less carbon atoms, which may be substituted with a hydroxy group.

Examples of the ester oil represented by the general formula (2) include at least one selected from the group consisting of glyceryl tri-2-ethylhexanoate and glyceryl tricaprylate. These may also be a plant-derived ester oil, such as jojoba oil, olive oil, sunflower oil, soybean oil, peanut oil, rapeseed oil, almond oil, palm oil, coconut oil, castor oil, wheat germ oil, grape seed oil, thistle oil, evening primrose oil, macadamia nut oil, corn germ oil, and avocado oil.

R⁷O-(AO)ₘ—COR⁸ (3)

In the general formula (3), R⁷ represents an aromatic hydrocarbon group having 6 or more and 20 or less carbon atoms; R⁸ represents an alkyl group or alkenyl group having 1 or more and 23 or less carbon atoms; AO represents an alkyleneoxy group having 2 or more and 4 or less carbon atoms; and m represents an average addition molar number of 1 or more and 50 or less.

R⁷ is an aromatic hydrocarbon group having preferably 6 or more carbon atoms from the viewpoint of improving the repellent sustaining effect, and preferably 12 or less carbon atoms, and more preferably 10 or less carbon atoms from the viewpoint of facilitating a feeling on the occasion of applying onto the skin, and R⁷ is still more preferably a benzyl group.

R⁸ is an alkyl group having preferably 7 or more carbon atoms, and more preferably 11 or more carbon atoms from the viewpoint of improving the repellent sustaining effect, and preferably 21 or less carbon atoms, and more preferably 15 or less carbon atoms from the viewpoint of facilitating a feeling on the occasion of applying onto the skin.

The AO group is preferably a propyleneoxy group from the viewpoint of improving the repellent sustaining effect; and m is preferably 1 or more and 10 or less, and more preferably 1 or more and 5 or less from the viewpoint of improving the repellent sustaining effect and the viewpoint of facilitating a feeling on the occasion of applying onto the skin.

Examples of the ester oil represented by the general formula (3) include at least one selected from the group consisting of an ester of a propylene oxide 3 mol adduct of benzyl alcohol and myristic acid (CRODAMOL STS, manufactured by Croda) and an ester of a propylene oxide 3 mol adduct of benzyl alcohol and 2-ethylhexanoic acid (CRODAMOL SFX, manufactured by Croda).

R⁹—O—(CH₂CH₂O)ᵥ—CO—(OCH₂CH₂)_{w}—OR¹⁰ (4)

In the general formula (4), R⁹ and R¹⁰ each independently represent an alkyl group or alkenyl group having 6 or more and 22 or less carbon atoms; and v and w are each independently 0 or an average addition molar number of 1 or more and 50 or less.

R⁹ and R¹⁰ are each an alkyl group having preferably 8 or more carbon atoms from the viewpoint of improving the repellent sustaining effect, and preferably 18 or less carbon atoms, and more preferably 12 or less carbon atoms from the viewpoint of facilitating a feeling on the occasion of applying onto the skin.

v and w are each preferably 0 or a number of 1 or more and 5 or less, and more preferably 0 from the viewpoint of facilitating a feeling on the occasion of applying onto the skin.

Examples of the dialkyl carbonate compound represented by the general formula (4) include dioctyl carbonate (Cetiol CC, manufactured by Cognis).

Examples of ester oils other than those mentioned above include an ester of a polyvalent carboxylic acid and an alcohol; and an ester of a polyhydric alcohol excluding glycerin and a fatty acid.

Specific examples thereof include at least one selected from the group consisting of diisopropyl dimerate, diisopropyl adipate, diethoxyethyl succinate, 2-ethylhexyl succinate, propanediol dicaprate, neopentyl glycol dicaprate, and neopentyl glycol di-2-ethylhexanoate. Of these, an ester of neopentyl glycol and a fatty acid is preferred, and at least one selected from neopentyl glycol dicaprate and neopentyl glycol di-2-ethylhexanoate is more preferred from the viewpoint of improving the repellent sustaining effect.

Of the aforementioned ester oils (b), at least one selected from the group consisting of the ester oil represented by the general formula (1) and an ester of neopentyl glycol and a fatty acid is preferred.

### (c) Ether Oil

As the ether oil, a dialkyl ether compound represented by the following general formula (5) or a polyoxyalkylene alkyl ether compound represented by the following general formula (6) is preferred from the viewpoint of improving the repellent sustaining effect.

R¹¹—O—R¹² (5)

In the general formula (5), R¹¹ and R¹² each independently represent a linear or branched alkyl group or alkenyl group having 6 or more and 22 or less carbon atoms or an aromatic hydrocarbon group having 6 or more and 24 or less carbon atoms.

R¹¹ and R¹² are each preferably an alkyl group from the viewpoint of improving the repellent sustaining effect, and the carbon number thereof is preferably 8 or more from the viewpoint of improving the repellent sustaining effect, and it is preferably 18 or less, more preferably 16 or less, and still more preferably 12 or less from the viewpoint of facilitating a feeling on the occasion of applying onto the skin.

Examples of the dialkyl ether compound represented by the general formula (5) include dihexyl ether, dioctyl ether (Cetiol OE, manufactured by Cognis), dicaprylyl ether, and cetyl-1,3-dimethylbutyl ether (ASE-166K, manufactured by Kao Corporation).

R¹³—O—(PO)ᵣ(EO)ₛ—H (6)

In the general formula (6), R¹³ represents an alkyl group or alkenyl group having 6 or more and 22 or less carbon atoms; PO represents a propyleneoxy group; EO represents an ethyleneoxy group; r is an average addition molar number of 0.1 or more and 15 or less; and s is an average addition molar number of 0 or more and 10 or less. In the case where s is not 0, an addition form of each of PO and EO may be a random form or may be a block form.

The carbon number of R¹³ is preferably 8 or more from the viewpoint of improving the repellent sustaining effect, and it is preferably 20 or less, more preferably 18 or less, and still more preferably 12 or less from the viewpoint of facilitating a feeling on the occasion of applying onto the skin.

The average addition molar number r is preferably 1 or more, more preferably 2 or more, and still more preferably 3 or more from the viewpoint of improving the repellent sustaining effect, and it is preferably 13 or less, and more preferably 10 or less from the viewpoint of facilitating a feeling on the occasion of applying onto the skin; and the average addition molar number s is preferably 5 or less, more preferably 1 or less, and still more preferably 0 from the viewpoint of facilitating a feeling on the occasion of applying onto the skin.

As the polyoxyalkylene alkyl ether compound represented by the general formula (6), at least one selected from the group consisting of polypropylene glycol; and polyoxypropylene octyl ether, polyoxypropylene decyl ether, and polyoxypropylene lauryl ether, in which the average addition molar number r of the propyleneoxy group is 3 or more and 10 or less, is preferred.

Of the aforementioned ether oils (c), the ether oil represented by the general formula (5) is preferred from the viewpoint of improving the repellent sustaining effect.

### (d) Hydrocarbon Oil

Examples of the hydrocarbon oil include a liquid paraffin, a liquid isoparaffin, squalane, isohexadecane, isoeicosane, hydrogenated polyisobutene, a light liquid isoparaffin, a heavy liquid isoparaffin, an α-olefin oligomer, and a cycloparaffin.

Of these, at least one selected from the group consisting of a liquid paraffin, a liquid isoparaffin, and squalane is preferred from the viewpoint of improving the repellent sustaining effect and the viewpoint of facilitating a feeling on the occasion of applying onto the skin.

### (e) Aliphatic Alcohol

Examples of the aliphatic alcohol include a monohydric chain or cyclic aliphatic alcohol, and from the viewpoint of improving the repellent sustaining effect, a monohydric chain aliphatic alcohol is preferred. The aliphatic alcohol is preferably an aliphatic alcohol having 14 or more carbon atoms, and more preferably 18 or more carbon atoms from the viewpoint of improving the repellent sustaining effect, and preferably 28 or less carbon atoms, more preferably 24 or less carbon atoms, and still more preferably 22 or less carbon atoms from the viewpoint of facilitating a feeling on the occasion of applying onto the skin.

Although the aforementioned aliphatic alcohol may be any of a chain aliphatic alcohol and a branched aliphatic alcohol, and it may be any of a saturated aliphatic alcohol and an unsaturated aliphatic alcohol, it is preferably a branched aliphatic saturated alcohol from the viewpoint of improving the repellent sustaining effect and the viewpoint of facilitating a feeling on the occasion of applying onto the skin.

Examples of the linear aliphatic alcohol include oleyl alcohol, and examples of the branched aliphatic saturated alcohol include butyl octanol, butyl decanol, hexyl decanol, and octyl dodecanol.

Of these, a branched aliphatic saturated alcohol is preferred, and at least one selected from the group consisting of hexyl decanol and octyl dodecanol is preferred from the viewpoint of improving the repellent sustaining effect and the viewpoint of facilitating a feeling on the occasion of applying onto the skin.

### (f) Polyhydric Alcohol

Examples of the polyhydric alcohol include an aliphatic alcohol having 2 or more carbon atoms, an aromatic alcohol, and a sugar alcohol having 4 or more carbon atoms, and the polyhydric alcohol may be either saturated or unsaturated. However, at least one selected from the group consisting of an aliphatic alcohol having 2 or more and 10 or less carbon atoms, an aromatic alcohol, and a sugar alcohol having 4 or more and 10 or less carbon atoms is preferred.

Among the polyhydric alcohols, examples of the aliphatic alcohol having 2 or more carbon atoms include dihydric alcohols, such as ethylene glycol, propylene glycol, 1,2-butylene glycol, 1,3-butylene glycol, 1,2-pentanediol, 1,2-hexanediol, diethylene glycol, dipropylene glycol, and hexylene glycol.

Examples of the aforementioned sugar alcohol include sorbitol, erythritol, pentaerythritol, xylitol, and mannitol.

Of the aforementioned liquid oily components, from the viewpoint of improving the repellent sustaining effect, at least one selected from the group consisting of the silicone oil (a), the ester oil (b), the ether oil (c), and the hydrocarbon oil (d) is preferred; at least one selected from the group consisting of the silicone oil (a), the ester oil (b), and the hydrocarbon oil (d) is more preferred; at least one selected from the group consisting of the silicone oil (a) and the ester oil (b) is still more preferred; and the silicone oil (a) is yet still more preferred. Of the silicone oils (a), from the viewpoint of improving the repellent sustaining effect, at least one selected from the group consisting of dimethylpolysiloxane, dimethiconol, methylphenylpolysiloxane, and modified silicone is preferred; at least one selected from the group consisting of dimethylpolysiloxane, dimethiconol, and polyether-modified silicone is more preferred; dimethylpolysiloxane is still more preferred; and linear dimethylpolysiloxane is yet still more preferred.

### (Content of Component (A))

The content of the component (A) in the composition of the present invention is 13% by mass or more, preferably 20% by mass or more, and more preferably 25% by mass or more from the viewpoint of improving the repellent sustaining effect and the viewpoint of facilitating a feeling on the occasion of applying onto the skin, and it is 50% by mass or less, preferably 40% by mass or less, and more preferably 35% by mass or less from the viewpoint of facilitating a feeling on the occasion of applying onto the skin and the viewpoint of securing the long-term storage stability of the composition. Then, the content of the component (A) in the composition of the present invention is 13 to 50% by mass, preferably 20 to 40% by mass, more preferably 25 to 40% by mass, and sill more preferably 25 to 35% by mass.

### <Component (B): Hydrophobic Particles>

The composition of the present invention contains, as the component (B), hydrophobic particles having a volume median particle diameter of 0.1 μm or more and 40 μm or less. In view of the fact that the composition of the present invention contains the component (B), the repellent sustaining effect due to the component (A) is improved, and further, when the composition is applied onto the skin, the feeling becomes good.

In this specification, the “hydrophobic particles” mean those having a wetting tension at 23° C. on the particle surface of 70 mN/m or less. In addition, the term “hydrophilic particles” means particles in which the foregoing wetting tension on the particle surface is more than 70 mN/m.

In view of the fact that the particles of the component (B) are hydrophobic particles having a specified particle diameter, a composition in which not only the repellent sustaining effect is improved, but also the feeling on the occasion of applying onto the skin is good is obtained. As for the component (B) which is used for the composition of the present invention, the wetting tension at 23° C. is preferably 60 mN/m or less, and more preferably 50 mN/m or less from the viewpoint of improving the repellent sustaining effect and the viewpoint of facilitating a feeling on the occasion of applying onto the skin. The wetting tension on the particle surface can be measured by the method described in the section of Examples.

The hydrophobic particles of the component (B) may be any of an organic particle, an inorganic particle, and a mixture thereof so long as they have the aforementioned hydrophobicity, and those which are typically blended in cosmetics can be used. Of these, hydrophobic organic particles are preferably contained in the component (B) from the viewpoint of improving the repellent sustaining effect, the viewpoint of facilitating a feeling on the occasion of applying onto the skin, and the viewpoint of securing the long-term storage stability of the composition.

Examples of a resin which constitutes the hydrophobic organic particles include a resin constituted of an acrylic resin, a silicone resin, a polystyrene resin, a polyamide resin, a polyester resin, a polyolefin resin, a polystyrene resin, a polyurethane resin, a vinyl resin, a urea resin, a phenol resin, a fluorine resin, a melamine resin, an epoxy resin, a polycarbonate resin, an acrylic-silicone copolymer resin, an acrylic-styrene copolymer resin, cellulose or the like. These hydrophobic organic resins may be either a crosslinking type resin particle or a non-crosslinking type resin particle. In addition, the hydrophobic organic particles may also be particles in which the surface of resin particle constituted of the aforementioned resin has been subjected to a hydrophobization treatment.

Of those mentioned above, from the viewpoint of improving the repellent sustaining effect and the viewpoint of facilitating a feeling on the occasion of applying onto the skin, resin particles constituted of at least one resin selected from the group consisting of a silicone resin, an acrylic resin, a polyurethane resin, and cellulose are preferred; resin particles constituted of at least one resin selected from the group consisting of a silicone resin, an acrylic resin, and a polyurethane resin are more preferred; and resin particles constituted of at least one resin selected from the group consisting of an acrylic resin and a silicone resin are still more preferred.

Examples of the acrylic resin constituting the hydrophobic organic particles include a homopolymer or a copolymer of an acrylic monomer. Examples of the acrylic monomer include (meth)acrylic acid and a (meth)acrylic acid ester. The term “(meth)acrylic acid” refers to both acrylic acid and methacrylic acid.

Among the (meth)acrylic acid esters, from the viewpoint of more enhancing the hydrophobicity, an alkyl (meth)acrylate is preferred, and the carbon number of the alkyl is preferably 1 or more and 18 or less, and more preferably 1 or more and 16 or less.

Specific examples of the alkyl (meth)acrylate include methyl (meth)acrylate, ethyl (meth)acrylate, n-butyl (meth)acrylate, isobutyl (meth)acrylate, sec-butyl (meth)acrylate, tert-butyl (meth)acrylate, 2-ethylhexyl (meth)acrylate, isodecyl (meth)acrylate, lauryl (meth)acrylate, and hexadecyl (meth)acrylate. These can be used alone or in combination of two or more thereof.

Besides the aforementioned (meth)acrylic acid and alkyl (meth)acrylates, the acrylic resin may be a crosslinking type acrylic resin resulting from further copolymerizing a polyfunctional (meth)acrylate, such as ethylene glycol di(meth)acrylate, propylene glycol di(meth)acrylate, and hexanediol di(meth)acrylate.

Specifically, examples of the acrylic resin include polymethyl methacrylate and an acrylic acid/methyl acrylate copolymer, and examples of the crosslinking type acrylic resin include a butyl acrylate/ethylene glycol dimethacrylate/sodium methacrylate copolymer and a lauryl methacrylate/ethylene glycol dimethacrylate/sodium methacrylate copolymer (hereinafter occasionally expressed as “lauryl methacrylate/Na methacrylate) crosspolymer”).

Of the hydrophobic organic particles, from the viewpoint of facilitating a feeling on the occasion of applying onto the skin, the acrylic resin particles are preferably particles composed of at least one acrylic resin selected from the group consisting of polymethyl methacrylate and a (lauryl methacrylate/Na methacrylate) crosspolymer. Examples of a commercially available product of the acrylic resin particles include particles described as a “crosslinked (meth)acrylic acid ester-based resin powder” in JP 2006-225311 A and GANZPEARL Series, manufactured by Aica Kogyo Co., Ltd.

Examples of the silicone resin constituting the hydrophobic organic particles include an organopolysiloxane, such as methyl polysiloxane and methylphenyl polysiloxane; a polyorganosilsesquioxane, such as polymethylsilsesquioxane; and composite materials thereof; and a copolymer containing an organopolysiloxane chain. Of these, from the viewpoint of improving the repellent sustaining effect and facilitating a feeling on the occasion of applying onto the skin, at least one selected from the group consisting of methyl polysiloxane, polymethylsilsesquioxane, and composite materials thereof is preferred.

In addition, a crosslinking type silicone resin resulting from crosslinking the aforementioned silicone resin with divinyl dimethyl polysiloxane (vinyl dimethicone), phenyl vinyl dimethyl polysiloxane (phenyl vinyl dimethicone), or the like can also be used. Examples of the crosslinking type silicone resin include a (dimethicone/vinyl dimethicone) crosspolymer, a (vinyl dimethicone/methiconesilsesquioxane) crosspolymer, and a (dimethicone/phenyl vinyl dimethicone) crosspolymer.

Of the hydrophobic organic particles, the silicone resin particles are preferably crosslinking type silicone resin particles, and more preferably particles composed of at least one silicone resin selected from the group consisting of a (dimethicone/vinyl dimethicone) crosspolymer and a (vinyl dimethicone/methiconesilsesquioxane) crosspolymer from the viewpoint of facilitating a feeling on the occasion of applying onto the skin.

Examples of a commercially available product of the silicone resin particles include KSP Series, KMP Series, “KSG-16”, and “Silicone X-52-1621”, all of which are manufactured by Shin-Etsu Chemical Co., Ltd.

From the viewpoint of improving the repellent sustaining effect and facilitating a feeling on the occasion of applying onto the skin, preferred examples of the hydrophobic inorganic particles include particles in which the surface of an inorganic particle of silica, alumina, talc, kaolin, clay, bentonite, mica, zirconium oxide, magnesium oxide, titanium oxide, zinc oxide, calcium sulfate, barium sulfate, magnesium sulfate, calcium carbonate, magnesium carbonate, or the like has been subjected to a hydrophobization treatment.

The shape of the hydrophobic particles of the component (B) is not particularly limited, and examples thereof include a spherical shape, a plate-like shape, a columnar shape, and an acicular shape. Of these, spherical particles are preferred from the viewpoint of improving the repellent sustaining effect and the viewpoint of facilitating a feeling on the occasion of applying onto the skin. In addition, particles having irregularities on the surface thereof and porous particles may also be used.

As for the hydrophobic particles of the component (B), from the viewpoint of facilitating a feeling on the occasion of applying onto the skin and the viewpoint of securing the long-term storage stability of the composition, the volume median particle diameter (D50) is 0.1 μm or more, preferably 0.5 μm or more, more preferably 1.0 μm or more, still more preferably 1.5 μm or more, and yet still more preferably 3.0 μm or more. In addition, from the viewpoint of improving the repellent sustaining effect and the viewpoint of facilitating a feeling on the occasion of applying onto the skin, the volume median particle diameter is 40 μm or less, preferably 30 μm or less, more preferably 27 μm or less, still more preferably 25 μm or less, and yet still more preferably 20 μm or less. Then, the volume median particle diameter of the component (B) is 0.1 to 40 μm, preferably 0.5 to 30 μm, more preferably 1.0 to 27 μm, still more preferably 1.5 to 25 μm, and yet still more preferably 3.0 to 20 μm.

The volume median particle diameter (D50) as referred to in the present invention means a particle diameter at 50% when calculated from particle diameters of smaller particle diameters in the cumulative volume frequency calculated in in terms of a volume fraction.

In the case of using two or more kinds of particles as the component (B), the volume median particle diameter of the component (B) means a volume median particle diameter of a mixture of all particles, and specifically, it can be determined according to the following equation (a).

Volume median particle diameter (D50)=Σ((Xi·Pi_{D50})/100ρi) (a)

Pi_{D50}: Volume median particle diameter (D50) of the particle group Pi constituting the particles (μm)

Xi: Mass ratio of the particle group Pi constituting the particles (%)

ρi: Density of the particle group Pi constituting the particles (g/cm³)

For example, the density of the resin particles is 1.18 g/cm³, the density of silica is 2.20 g/cm³, and the density of titanium oxide is 4.17 g/cm³.

The volume median particle diameter can be measured using a laser scattering particle size distribution analyzer, and specifically, it can be measured by the method described in the section of Examples.

As for the component (B) in the present invention, from the viewpoint of improving the repellent sustaining effect and facilitating a feeling on the occasion of applying onto the skin, in a distribution curve of the particle diameters of the component (B) as measured using a laser scattering particle size distribution analyzer, a volume proportion of particle diameters of 45 μm or more relative to the volume of the whole of particles is preferably 15% or less, and more preferably 10% or less. In view of the fact that the composition of the present invention contains particles having such a particle size distribution, structural viscosity is imparted to the composition, and it becomes possible to improve a repellent sustaining time while facilitating a feeling on the occasion of applying onto the skin. Specifically, the particle size distribution of the component (B) can be measured by the method described in the section of Examples.

### (Content of Component (B))

From the viewpoint of improving the repellent sustaining effect and the viewpoint of facilitating a feeling on the occasion of applying onto the skin, the content of the component (B) in the composition of the present invention is 0.5% by mass or more, preferably 2% by mass or more, more preferably 4% by mass or more, still more preferably 6% by mass or more, yet still more preferably 7% by mass or more, and especially preferably 8% by mass or more. In addition, from the viewpoint of improving the repellent sustaining effect, it is 35% by mass or less, preferably 27% by mass or less, more preferably 18% by mass or less, still more preferably 16% by mass or less, and yet still more preferably 13% by mass or less. Then, the content of the component (B) in the composition of the present invention is 0.5 to 35% by mass, preferably 2 to 27% by mass, more preferably 4 to 27% by mass, still more preferably 6 to 18% by mass, yet still more preferably 7 to 16% by mass, even yet still more preferably 7 to 13% by mass, and especially preferably 8 to 13% by mass.

### <Component (C): Water>

The composition of the present invention contains water as the component (C). The content of the component (C) in the composition is 30% by mass or more from the viewpoint of providing an oil-in-water type composition, and it is preferably 40% by mass or more, more preferably 45% by mass or more, and still more preferably 50% by mass or more from the viewpoint of facilitating a feeling on the occasion of applying onto the skin. In addition, from the viewpoint of improving the repellent sustaining effect and the viewpoint of facilitating a feeling on the occasion of applying onto the skin, it is 86.5% by mass or less, preferably 78% by mass or less, more preferably 70% by mass or less, and still more preferably 65% by mass or less. A specific range of the content of the component (C) in the composition is 30 to 86.5% by mass, preferably 40 to 78% by mass, more preferably 40 to 70% by mass, still more preferably 40 to 65% by mass, yet still more preferably 45 to 65% by mass, and even yet still more preferably 50 to 65% by mass. An upper limit of the total amount of the components (A) to (C) is 100% by mass.

In the composition of the present invention, from the viewpoint of improving the repellent sustaining effect, the viewpoint of facilitating a feeling on the occasion of applying onto the skin, and the viewpoint of securing the long-term storage stability, a mass ratio [(A)/(B)] of the component (A) to the component (B) is preferably 1 or more, more preferably 1.3 or more, still more preferably 1.5 or more, and yet still more preferably 2 or more, and from the same viewpoints, it is preferably 50 or less, more preferably 30 or less, still more preferably 10 or less, yet still more preferably 7 or less, and even yet still more preferably 5 or less.

In the composition of the present invention, from the viewpoint of improving the repellent sustaining effect, the viewpoint of facilitating a feeling on the occasion of applying onto the skin, and the viewpoint of securing the long-term storage stability, a mass ratio [(A)/(C)] of the component (A) to the component (C) is preferably 0.15 or more, more preferably 0.2 or more, still more preferably 0.3 or more, and yet still more preferably 0.4 or more, and from the same viewpoints, it is preferably 1.5 or less, more preferably 1.2 or less, still more preferably 1.0 or less, and yet still more preferably 0.8 or less.

### (Surfactant)

The composition of the present invention can further contain a surfactant from the viewpoint of providing an oil-in-water type composition. As the surfactant, known surfactants can be used, and any of an anionic surfactant, a cationic surfactant, an ampholytic surfactant, a nonionic surfactant, and so on can be used. These can be used alone or in combination of two or more thereof.

From the viewpoint of providing an oil-in-water type composition, the content of the surfactant in the composition of the present invention is preferably 0.01% by mass or more, more preferably 0.05% by mass or more, and still more preferably 0.1% by mass or more, and from the same viewpoint, it is preferably 5% by mass or less, more preferably 3% by mass or less, and still more preferably 1% by mass or less.

### (Component (D): Thickener)

The composition of the present invention can further contain a thickener as the component (D). As the thickener, one having an appropriate emulsifying ability (namely, surface active ability) is preferred from the viewpoint of regulating fluidity after applying onto the skin, to improve the repellent sustaining effect, the viewpoint of allowing each of the components to well disperse or dissolve, to make it possible to secure excellent coatability onto the skin, and the viewpoint of securing long-term storage stability of the composition. In the present invention, an agent having an emulsifying ability and a thickening effect is the thickener. Specifically, examples of the component (D) include water-soluble cationic polymers, anionic polymers, nonionic polymers, and amphoteric polymers or dipolar polymers.

From the viewpoint of obtaining the aforementioned effects, the content of the component (D) in the composition of the present invention is preferably 0.01% by mass or more, more preferably 0.05% by mass or more, and still more preferably 0.1% by mass or more, and from the same viewpoint, it is preferably 5% by mass or less, more preferably 3% by mass or less, and still more preferably 1% by mass or less. In the case where the composition of the present invention contains the thickener having a surface active ability, the content of other surfactant is preferably 1.5% by mass or less, and more preferably 1% by mass or less.

### <Other Components>

The composition of the present invention can also contain other components than those as mentioned above, for example, medicinal ingredients or physiologically active ingredients, such as a preservative, a coloring agent, a moisturizer, a fragrance, a pH modifier, a vitamin, a blood circulation accelerator, an active oxygen scavenger, an anti-inflammatory agent, a whitening agent, and a disinfectant, within a range where the effects of the present invention are not impaired. Each of these components can also be used for other applications, and for example, the fragrance is used as the disinfectant.

From the viewpoint of preventing the user from occurrence of allergy or chopped skin, it is preferred that the composition of the present invention contains the component (A) as a repellent active component for pest repellence and does not contain an effective amount of other pest repellent.

Even when the composition of the present invention does not contain an effective amount of an existing pest repellent as mentioned later, it has the repellent sustaining effect. In other words, even in the case where the composition of the present invention contains an pest repellent other than the component (A) in an amount of less than the lower limit of the effective amount, so long as a pest lands but does not stay on that place, the effect of preventing the pest from staying is developed. That is, the composition of the present invention may not substantially contain a pest repellent other than the component (A).

Here, the phrase “does not contain an effective amount” of a pest repellent other than the component (A) generally means that the content of an existing pest repellent than the component (A) is preferably 15% by mass or less, more preferably 10% by mass or less, still more preferably 5% by mass or less, yet still more preferably 4% by mass or less, even yet still more preferably 3% by mass or less, and even still more preferably 1% by mass or less in the composition of the present invention.

As for the effective amount of the existing pest repellent, for example, the minimum effective amount and the like published by the manufacturer and the like of each repellent product can be made by reference.

More specifically, the effective amount of DEET: N,N-diethyl-toluamide is 4% by mass or more, and preferably 10% by mass or more; the effective amount of Icaridin: 1-methylpropyl-2-(2-hydroxyethyl)-1-piperidine carboxylate is 4% by mass or more; the effective amount of IR3535: ethyl 3-(acetylbutyl)aminopropionate is 4% by mass or more; and the effective amount of citronella oil is 10% by mass or more, each of these compounds being an existing pest repellent as mentioned later.

The effective amount of the existing pest repellent can also be measured by a repellent evaluation test as mentioned below.

### (Repellent Evaluation Test)

100 female mosquitoes (Aedes albopictus) that have been mated are placed in a plastic cage (30×30×30 cm: BugDorm-1 cage) surrounded by a mesh. An arm is inserted into a KUALATEC Super Long Glove (50 cm) (available from AS ONE Corporation, Catalog No.: 3-6432-02) with a cut of a rectangular shape having a size of 5 cm in length×4 cm in width on the elbow side of about 15 cm from the wrist. The arm is inserted into the cage in a state that nothing is applied to an exposed skin portion from the cut, and it is confirmed that the mosquitoes land in two locations of the exposed skin portion within 2 minutes and then stay for at least 1 second. In the case where the mosquito does not stay for at least one second, a new mosquito is prepared. The “stay for at least 1 second” is hereinafter referred to simply as “stay”.

The test of an evaluation sample that is a solution of the pest repellent active ingredient is performed by adjusting the concentration thereof with ethanol such that it can be applied at a coverage of 2 mg/cm² to the exposed skin portion (5 cm×4 cm).

Using a pipetman, the solution whose concentration has been adjusted is placed in the exposed skin portion and applied so as to spread over the entire exposed skin portion (required solution volume: 40 to 50 μL). Subsequently, after allowing to stand for 3 minutes, the test is started.

The test is performed by inserting the arm applied with the evaluation sample into the cage for 2 minutes and counting the number of stays. The test is terminated when the vehicle has stayed twice in total, and the test is performed in which the arm is inserted for 2 minutes every 30 minutes until the test is completed. In the case where there is a second stay at 30 minutes, the repellent effect duration is determined to be 0 minute, and in the case where there is a second stay at 60 minutes, the repellent effect duration is determined to be 30 minutes. The test is performed on three subjects, and an average repellent effect duration is calculated.

In the present test, the concentration of the pest repellent at which an average repellent effect duration of 2 hours or more in average is exhibited can be taken as the effective concentration (effective amount) of the pest repellent.

In view of the fact that even when the composition of the present invention does not contain an effective amount of an existing pest repellent, it has the repellent sustaining effect, it is rather preferred that the composition of the present invention does not substantially contain other pest repellent than the component (A). In the composition of the present invention, the content of the other pest repellent than the component (A) is preferably 0.5% by mass or less, more preferably 0.1% by mass or less, still more preferably 0.01% by mass or less, and yet still more preferably substantially 0% by mass.

Examples of the existing pest repellent other than the component (A) include known pest repellent compounds, such as DEET, Icaridin, dimethyl phthalate, 2-ethyl- 1,3-hexanediol, p-menthane-3,8-diol, karan-3,4-diol, di-n-butyl succinate, hydroxyanisole, rotenone, ethyl 3-(acetylbutyl)aminopropionate, citronellol, eucalyptol, α-pinene, geraniol, citronellal, camphor, linalool, and 2-undecanone, and besides, repellent refined oils, such as citronella oil and lemongrass oil. The “repellent refined oil” means one having a pest repellent effect among refined oils (essential oils) obtained by extraction, distillation, compression, or the like of a component contained in a plant.

The composition of the present invention can be produced by a conventional method. For example, the composition of the present invention can be produced by previously blending the components (B) and (C) and mixing using a stirring apparatus, such as a homogenizer, and then blending the component (A) and optionally other component, followed by stirring.

Although the pest targeted by the composition of the present invention is not particularly limited, the composition of the present invention is more effective for flying pests.

The “flying pests” refer to pests that approach animals, such as humans, while flying and suck blood from their skins, pests that carry pathogenic bacteria or the like while flying without sucking blood, and pests in which their own flying gives a feeling of displeasure to humans.

Specific examples of the flying pests include house mosquitoes, such as Culex pipiens pallens, Culex tritaeniorhynchus, Culex pipiens molestus, and Culex quinquefasciatus; mosquitoes, such as Aedes aegypti, Aedes albopictus, Anopheles sinensis, Aedes togoi, Anopheles gambiae, and Anopheles stephensi; chironomidae, such as Chironomus yoshimatsui and Propsilocerus akamusi; black flies, such as Twinnia japonensis, Prosimulium yezoense, and Odagmia aokii; flies, such as Musca domestica, Muscina stabulans, Fannia canicularis, Calliphoridae, Sarcophagidae, Delia platura, Delia antiqua, fruit flies, fruit vinegar flies, moth flies, tsetse flies, and Stomoxys calcitrans; horseflies, such as Tabanus stygius, Tabanus trigonus, Chrysops suavis, and Haematopota pluvialis; biting midges, such as Leptoconops nipponensis, Culicoides sumatrae, and Culicoides arakawae; and bees, such as Vespa simillima xanthoptera, Polistes jokahamae, and honeybees.

The composition of the present invention exhibits an excellent repellent sustaining effect particularly against mosquitoes among them. The composition of the present invention exhibits an excellent repellent sustaining effect against Culex pipiens pallens, Culex tritaeniorhynchus, Culex pipiens molestus, Culex quinquefasciatus, Aedes aegypti, and Aedes albopictus among the aforementioned mosquitoes.

### [Method for Repelling Pests and Method for Inhibiting Pest Stay]

The method for repelling pests of the present invention is a method including applying the pest repellent composition of the present invention onto the skin surface of a human.

The method for inhibiting pest stay of the present invention is a method including attaching the pest stay inhibition composition of the present invention onto limbs of a pest, particularly a flying pest, to prevent the pest from staying on the skin. These methods are also hereunder collectively referred to as “method of the present invention”.

Here, the phrase “applying onto the skin surface” includes not only directly applying the composition onto the skin surface by a finger or the like but also attaching the composition onto the skin surface by spraying or the like.

The amount of the composition which is applied onto the skin surface is preferably 0.1 mg or more, more preferably 0.2 mg or more, and still more preferably 0.25 mg or more per 1 cm² from the viewpoint of improving the repellent sustaining effect. In addition, an upper limit of the application amount is preferably 10 mg or less, more preferably 8 mg or less, and still more preferably 5 mg or less per 1 cm² from the viewpoint of suppression of stickiness and economy.

From the viewpoint of providing a repellent composition which is excellent in the repellent sustaining effect against a mosquito, is safe, is good in the feeling on the occasion of applying onto the skin, and is excellent in the long-term storage stability, the composition of the present invention is preferably an oil-in-water type mosquito repellent composition or mosquito stay inhibition composition containing the following components (A) to (C) and having the content of other pest repellent than the component (A) of 15% by mass or less:

(A) 13% by mass or more and 50% by mass or less of at least one non-volatile liquid oily component selected from the group consisting of a silicone oil, an ester oil, an ether oil, a hydrocarbon oil, an aliphatic alcohol, and a polyhydric alcohol, the non-volatile liquid oily component having a surface tension at 25° C. of 40 mN/m or less and a viscosity at 23° C. as measured with a B-type rotational viscometer of 400 mPa·s or less;

(B) 0.5% by mass or more and 35% by mass or less of hydrophobic particles having a volume median particle diameter of 0.1 μm or more and 40 μm or less; and

(C) 30% by mass or more and 86.5% by mass or less of water.

From the viewpoint of providing a repellent composition which is excellent in the repellent sustaining effect against a mosquito, is safe, is good in the feeling on the occasion of applying onto the skin, and is excellent in the long-term storage stability, the composition of the present invention is more preferably an oil-in-water type mosquito repellent composition or mosquito stay inhibition composition containing the following components (A) to (C) and having the content of other pest repellent than the component (A) of 15% by mass or less:

(A) 13% by mass or more and 50% by mass or less of at least one non-volatile liquid oily component selected from the group consisting of a silicone oil, an ester oil, an ether oil, a hydrocarbon oil, an aliphatic alcohol, and a polyhydric alcohol, the non-volatile liquid oily component having a surface tension at 25° C. of 40 mN/m or less and a viscosity at 23° C. as measured with a B-type rotational viscometer of 400 mPa·s or less;

(B) 0.5% by mass or more and 35% by mass or less of hydrophobic particles containing resin particles constituted of at least one resin selected from the group consisting of a silicone resin, an acrylic resin, and a polyurethane resin and having a volume median particle diameter of 0.1 μm or more and 40 μm or less; and

(C) 30% by mass or more and 86.5% by mass or less of water.

From the viewpoint of providing a repellent composition which is excellent in the repellent sustaining effect against a mosquito, is safe, is good in the feeling on the occasion of applying onto the skin, and is excellent in the long-term storage stability, the composition of the present invention is preferably an oil-in-water type mosquito repellent composition or mosquito stay inhibition composition containing the following components (A) to (C), with a mass ratio [(A)/(B)] of the component (A) to the composition (B) being 2 or more and 5 or less, and having the content of other pest repellent than the component (A) of 1% by mass or less:

(A) 13% by mass or more and 50% by mass or less of at least one non-volatile liquid oily component selected from the group consisting of a silicone oil, an ester oil, an ether oil, a hydrocarbon oil, an aliphatic alcohol, and a polyhydric alcohol, the non-volatile liquid oily component having a surface tension at 25° C. of 40 mN/m or less and a viscosity at 23° C. as measured with a B-type rotational viscometer of 400 mPa·s or less;

(B) 0.5% by mass or more and 35% by mass or less of hydrophobic particles having a volume median particle diameter of 1.5 μm or more and 25 μm or less; and

(C) 30% by mass or more and 86.5% by mass or less of water.

From the viewpoint of providing a repellent composition which is excellent in the repellent sustaining effect against a mosquito, is safe, is good in the feeling on the occasion of applying onto the skin, and is excellent in the long-term storage stability, the composition of the present invention is preferably an oil-in-water type mosquito repellent composition or mosquito stay inhibition composition containing the following components (A) to (D), with a mass ratio [(A)/(B)] of the component (A) to the composition (B) being 2 or more and 5 or less and a mass ratio [(A)/(C)] of the component (A) to the component (C) being 0.2 or more and 1.2 or less, and having the content of other pest repellent than the component (A) of 1% by mass or less:

(A) 13% by mass or more and 50% by mass or less of at least one non-volatile liquid oily component selected from the group consisting of a silicone oil, an ester oil, and a hydrocarbon oil, the non-volatile liquid oily component having a surface tension at 25° C. of 40 mN/m or less and a viscosity at 23° C. as measured with a B-type rotational viscometer of 400 mPa·s or less;

(B) 0.5% by mass or more and 35% by mass or less of hydrophobic particles having a volume median particle diameter of 1.5 μm or more and 25 μm or less;

(C) 30% by mass or more and 86.5% by mass or less of water; and

(D) 0.05% by mass or more and 3% by mass or less of a thickener.

In the aforementioned composition, the component (B) more preferably contains resin particles constituted of at least one resin selected from the group consisting of a silicone resin, an acrylic resin, and a polyurethane resin.

The method for repelling pests of the present invention is a method in which even if a pest, such as a mosquito, lands on the target onto which the composition of the present invention has been applied or attached, the pest does not stay on that place, whereby the pest is repelled. Specifically, the method for repelling pests of the present invention has an effect of preventing a pest, such as a mosquito, from staying on the body surface of an animal, such a human, after landing thereon, for a time enough to pierce the animal, specifically, for example, 1 second or more. Such an effect is based on an unprecedented pest repellent principle and is safe without causing side effects, such as chopped skin.

Regarding the aforementioned embodiments, the present invention further discloses the following embodiments.

<1>

An oil-in-water type pest repellent composition or pest stay inhibition composition containing the following components (A) to (C), with a mass ratio [(A)/(B)] of the component (A) to the composition (B) being 1 or more and 50 or less:

(A) 13% by mass or more and 50% by mass or less of at least one non-volatile liquid oily component selected from the group consisting of a silicone oil, an ester oil, an ether oil, a hydrocarbon oil, an aliphatic alcohol, and a polyhydric alcohol, the non-volatile liquid oily component having a surface tension at 25° C. of 40 mN/m or less and a viscosity at 23° C. as measured with a B-type rotational viscometer of 400 mPa·s or less;

(B) 0.5% by mass or more and 35% by mass or less of hydrophobic particles having a volume median particle diameter of 0.1 μm or more and 40 μm or less; and

(C) 30% by mass or more and 86.5% by mass or less of water.

<2>

The composition as set forth in <1>, wherein the mass ratio [(A)/(B)] of the component (A) to the composition (B) is preferably 1.3 or more, more preferably 1.5 or more, and still more preferably 2 or more, and it is preferably 30 or less, more preferably 10 or less, still more preferably 7 or less, and yet still more preferably 5 or less.

<3>

The composition as set forth in <1> or <2>, wherein the mass ratio [(A)/(C)] of the component (A) to the composition (C) is 0.15 or more, preferably 0.2 or more, more preferably 0.3 or more, and still more preferably 0.4 or more, and it is 1.5 or less, preferably 1.2 or less, more preferably 1.0 or less, and still more preferably 0.8 or less.

<4>

The composition as set forth in any one of <1> to <3>, wherein the content of other pest repellent than the component (A) is 15% by mass or less, preferably 10% by mass or less, more preferably 5% by mass or less, still more preferably 3% by mass or less, yet still more preferably 2% by mass or less, and even yet still more preferably 1% by mass or less.

<5>

The composition as set forth in any one of <1> to <4>, wherein the surface tension at 25° C. of the component (A) is 15 mN/m or more, and preferably 17 mN/m or more, and it is 30 mN/m or less, preferably 28 mN/m or less, more preferably 25 mN/m or less, still more preferably 23 mN/m or less, and yet still more preferably 21 mN/m or less.

<6>

The composition as set forth in any one of <1> to <5>, wherein the viscosity at 23° C. of the component (A) as measured with a B-type rotational viscometer is 1 mPa·s or more, and it is 300 mPa·s or less, preferably 210 mPa·s or less, more preferably 100 mPa·s or less, still more preferably 60 mPa·s or less, yet still more preferably 40 mPa·s or less, and even yet still more preferably 30 mPa·s or less.

<7>

The composition as set forth in any one of <1> to <6>, wherein the component (A) is at least one selected from the group consisting of a silicone oil, an ester oil, an ether oil, and a hydrocarbon oil, preferably at least one selected from the group consisting of a silicone oil, an ester oil, and a hydrocarbon oil, more preferably at least one selected from the group consisting of a silicone oil and an ester oil, and still more preferably a silicone oil.

<8>

The composition as set forth in any one of <1> to <7>, wherein the content of the component (A) is 20% by mass or more, and preferably 25% by mass or more, and it is preferably 40% by mass or less, and more preferably 35% by mass or less.

<9>

The composition as set forth in any one of <1> to <8>, wherein the component (B) contains preferably resin particles constituted of at least one resin selected from the group consisting of an acrylic resin, a silicone resin, a polystyrene resin, a polyamide resin, a polyester resin, a polyolefin resin, a polystyrene resin, a polyurethane resin, a vinyl resin, a urea resin, a phenol resin, a fluorine resin, a melamine resin, an epoxy resin, a polycarbonate resin, an acrylic-silicone copolymer resin, an acrylic-styrene copolymer resin, and cellulose; more preferably resin particles constituted of at least one resin selected from the group consisting of a silicone resin, an acrylic resin, a polyurethane resin, and cellulose; still more preferably resin particles constituted of at least one resin selected from the group consisting of a silicone resin, an acrylic resin, and a polyurethane resin; and yet still more preferably resin particles constituted of at least one resin selected from the group consisting of an acrylic resin and a silicone resin.

<10>

The composition as set forth in any one of <1> to <9>, wherein the content of the component (B) is preferably 2% by mass or more, more preferably 4% by mass or more, still more preferably 6% by mass or more, yet still more preferably 7% by mass or more, and especially preferably 8% by mass or more, and it is preferably 27% by mass or less, more preferably 18% by mass or less, still more preferably 16% by mass or less, and yet still more preferably 13% by mass or less.

<11>

The composition as set forth in any one of <1> to <10>, wherein the volume median particle diameter of the component (B) is preferably 0.5 μm or more, more preferably 1.0 μm or more, still more preferably 1.5 μm or more, and yet still more preferably 3.0 μm or more, and it is preferably 30 μm or less, more preferably 27 μm or less, still more preferably 25 μm or less, and yet still more preferably 20 μm or less.

<12>

The composition as set forth in any one of <1> to <11>, wherein in a distribution curve of particle diameters of the component (B) as measured using a laser scattering particle size distribution analyzer, a volume proportion of particle diameters of 45 μm or more relative to the volume of the whole of particles is 15% or less, and preferably 10% or less.

<13>

The composition as set forth in any one of <1> to <12>, wherein the content of the component (C) is 40% by mass or more, preferably 45% by mass or more, and more preferably 50% by mass or more, and it is 78% by mass or less, preferably 70% by mass or less, and more preferably 65% by mass or less.

<14>

The composition as set forth in any one of <1> to <13>, further containing, as a component (D), a thickener in an amount of preferably 0.01% by mass or more, more preferably 0.05% by mass or more, and still more preferably 0.1% by mass or more, and preferably 5% by mass or less, more preferably 3% by mass or less, and still more preferably 1% by mass or less.

<15>

The composition as set forth in <14>, containing, as the component (D), at least one selected from the group consisting of water-soluble cationic polymers, anionic polymers, nonionic polymers, and amphoteric polymers or dipolar polymers.

<16>

The composition as set forth in any one of <4> to <15>, wherein the other pest repellent than the component (A) is at least one selected from the group consisting of DEET, Icaridin, ethyl 3-(acetylbutyl)aminopropionate, and citronella oil.

<17>

An oil-in-water type mosquito repellent composition or mosquito stay inhibition composition containing the following components (A) to (C) and having the content of other pest repellent than the component (A) of 15% by mass or less:

(A) 13% by mass or more and 50% by mass or less of at least one non-volatile liquid oily component selected from the group consisting of a silicone oil, an ester oil, an ether oil, a hydrocarbon oil, an aliphatic alcohol, and a polyhydric alcohol, the non-volatile liquid oily component having a surface tension at 25° C. of 40 mN/m or less and a viscosity at 23° C. as measured with a B-type rotational viscometer of 400 mPa·s or less;

(B) 0.5% by mass or more and 35% by mass or less of hydrophobic particles containing resin particles constituted of at least one resin selected from the group consisting of a silicone resin, an acrylic resin, and a polyurethane resin and having a volume median particle diameter of 0.1 μm or more and 40 μm or less; and

(C) 30% by mass or more and 86.5% by mass or less of water.

<18>

An oil-in-water type mosquito repellent composition or mosquito stay inhibition composition containing the following components (A) to (C), with a mass ratio [(A)/(B)] of the component (A) to the composition (B) being 2 or more and 5 or less, and having the content of other pest repellent than the component (A) of 1% by mass or less:

(A) 13% by mass or more and 50% by mass or less of at least one non-volatile liquid oily component selected from the group consisting of a silicone oil, an ester oil, an ether oil, a hydrocarbon oil, an aliphatic alcohol, and a polyhydric alcohol, the non-volatile liquid oily component having a surface tension at 25° C. of 40 mN/m or less and a viscosity at 23° C. as measured with a B-type rotational viscometer of 400 mPa·s or less;

(B) 0.5% by mass or more and 35% by mass or less of hydrophobic particles having a volume median particle diameter of 1.5 μm or more and 25 μm or less; and

(C) 30% by mass or more and 86.5% by mass or less of water.

<19>

An oil-in-water type mosquito repellent composition or mosquito stay inhibition composition containing the following components (A) to (D), with a mass ratio [(A)/(B)] of the component (A) to the composition (B) being 2 or more and 5 or less and a mass ratio [(A)/(C)] of the component (A) to the component (C) being 0.2 or more and 1.2 or less, and having the content of other pest repellent than the component (A) of 1% by mass or less:

(A) 13% by mass or more and 50% by mass or less of at least one non-volatile liquid oily component selected from the group consisting of a silicone oil, an ester oil, and a hydrocarbon oil, the non-volatile liquid oily component having a surface tension at 25° C. of 40 mN/m or less and a viscosity at 23° C. as measured with a B-type rotational viscometer of 400 mPa·s or less;

(B) 0.5% by mass or more and 35% by mass or less of hydrophobic particles having a volume median particle diameter of 1.5 μm or more and 25 μm or less;

(C) 30% by mass or more and 86.5% by mass or less of water; and

(D) 0.05% by mass or more and 3% by mass or less of a thickener.

<20>

The oil-in-water type mosquito repellent composition or mosquito stay inhibition composition as set forth in <18> or <19>, wherein the component (B) contains resin particles constituted of at least one resin selected from the group consisting of a silicone resin, an acrylic resin, and a polyurethane resin.

<21>

A method for repelling pests, including applying the composition as set forth in any one of <1> to <20> onto the skin surface of a human.

<22>

A method for inhibiting pest stay, including attaching the composition as set forth in any one of <1> to <20> onto limbs of a pest to prevent the pest from staying on the skin of a human.

### EXAMPLES

The present invention is hereunder described by reference to Examples, but the present invention is not limited to the scope of the Examples. In the present Examples, various measurements and evaluations were performed by the following methods.

### (Surface Tension of Liquid Oily Component)

The surface tension of liquid oily component is a surface tension measured in an environment at 25° C. by the Wilhelmy method using a platinum plate, using an automatic surface tensiometer: Tensiometer K100 (manufactured by KRUSS GmbH).

### (Viscosity of Liquid Oily Component)

Viscometer TVB-10, manufactured by Told Sangyo Co., Ltd. was used as a B-type rotational viscometer according to JIS K7117-1:1999. As to components to be measured, a viscosity value greatly differs for every sample, and therefore, it is difficult to accurately measure all components under a single measurement condition. Then, the measurement was performed using two types of rotors. The viscosity was first measured at a rotational speed of 12 rpm using a rotor M2 in an environment at 25° C. At this time, the component having a viscosity of 2,500 mPa·s or more was again measured at a rotational speed of 6 rpm, to obtain the viscosity value.

On the other hand, the component having a viscosity of 20 mPa·s or less was again measured at a rotational speed of 30 rpm using an L adapter that is a rotor for low viscosity in an environment of 23° C., to obtain the viscosity value.

### (Wetting Tension on the Particle Surface)

Using a wetting tension test mixture of FUJIFILM Wako Pure Chemical Corporation, the measurement was performed in the following manner.

0.2 mL of a wetting tension test mixture No. 70 (surface tension: 70 mN/m) was weighed in a vessel in an environment at 23° C. Then, 0.01 g of particles to be measured were slowly added in the aforementioned mixture.

30 seconds after adding the particles, whether or not the particles had been wetted with the aforementioned mixture was visually confirmed, and in the case where the particles had been wetted, the wetting tension on the particle surface was judged to be 70 mN/m or less. Since the surface tension of water is 70 mN/m, when the wetting tension on the particle surface was 70 mN/m or less, the particles were judged to be hydrophobic.

What the particle surface is wetted with the aforementioned mixture means that the mixture permeates into the particles. The state in which the particles are not wetted with the mixture means that even when the particles and the mixture are mixed with each other, the particles are separated (or repelled) from the mixture.

The wetting tension on each of the particle surfaces was measured by performing the aforementioned test using each of the following wetting tension test mixtures. For example, what the surface tension on the particle surface is 22.6 mN/m means that in the aforementioned test, when using No. 25.4 (surface tension: 25.4 mN/m) as the wetting tension test mixture, the particle surface was not wetted with the foregoing mixture, whereas when using No. 22.6 (surface tension: 22.6 mN/m) as the wetting tension test mixture, the particle surface was wetted with the foregoing mixture.

### <Wetting Tension Test Mixtures of FUJIFILM Wako Pure Chemical Corporation>

No. 70 (surface tension: 70 mN/m), No. 67 (surface tension: 67 mN/m), No. 64 (surface tension: 64 mN/m), No. 62 (surface tension: 62 mN/m), No. 60 (surface tension: 60 mN/m), No. 59 (surface tension: 59 mN/m), No. 52 (surface tension: 52 mN/m), No. 50 (surface tension: 50 mN/m), No. 48 (surface tension: 48 mN/m), No. 46 (surface tension: 46 mN/m), No. 44 (surface tension: 44 mN/m), No. 42 (surface tension: 42 mN/m), No. 40 (surface tension: 40 mN/m), No. 38 (surface tension: 38 mN/m), No. 36 (surface tension: 36 mN/m), No. 34 (surface tension: 34 mN/m), No. 32 (surface tension: 32 mN/m), No. 30 (surface tension: 30 mN/m), No. 27.3 (surface tension: 27.3 mN/m), No. 25.4 (surface tension: 25.4 mN/m), and No. 22.6 (surface tension: 22.6 mN/m)

### (Volume Median Particle Diameter)

With respect to particles of (B2) to (B10), (b2), and (b3) shown in Table 1, the volume median particle diameter (D50) of particles was measured using a laser scattering particle size distribution analyzer “LA-920”, manufactured by Horiba, Ltd. while using ethanol/water (99.5% by mass/0.5% by mass) as a dispersion medium. In addition, a relative refractive index of 1.05-0.000i was used on the occasion of measuring the particles of (B2), (B4), (B7), (B9), and (b2), and a relative refractive index of 1.10-0.000i was used on the occasion of measuring the particles of (B3), (B5), (B6), and (B8).

In addition, with respect to particles of (B1) and (b1) shown in Table 1, the measurement was performed using a dynamic light scattering particle size distribution analyzer “LB-500”, manufactured by Horiba, Ltd. With respect to particles of (B1), the measurement was performed at a relative refractive index of 1.05-0.000i by using Silicone 6cs (A1) of the component (A). In addition, with respect to particles of (b1), the measurement was performed at a relative refractive index of 2.00-0.000i by using 2-ethylhexyl p-methoxycinnamate (“Uvinul MC80”, manufactured by BASF SE) as a dispersion medium. In addition, as already mentioned, in the case of using two or more kinds of particles, the volume median particle diameter was measured according to the following equation (a).

Volume median particle diameter (D50)=Σ((Xi·Pi_{D50})/100ρi) (a)

Pi_{D50}: Volume median particle diameter (D50) of the particle group Pi constituting the particles (μm)

Xi: Mass ratio of the particle group Pi constituting the particles (%)

ρi: Density of the particle group Pi constituting the particles (g/cm³)

The density of the resin particles was defined to be 1.18 g/cm³, the density of silica was defined to be 2.20 g/cm³, and the density of titanium oxide was defined to be 4.17 g/cm³.

### (Particle Size Distribution)

With respect to particles of (B2) to (B9) shown in Table 1, the particle size distribution of particles was measured using a laser scattering particle size distribution analyzer (“LA-920”, manufactured by Horiba, Ltd., measuring condition: particle diameter basis (volume)) while using ethanol/water (99.5% by mass/0.5% by mass) as a dispersion medium. A relative refractive index of 1.05-0.000i was used on the occasion of measuring the particles of (B2), (B4), (B7), and (B9), and a relative refractive index of 1.10-0.000i was used on the occasion of measuring the particles of (B3), (B5), (B6), and (B8). On that occasion, in a chart obtained through the measurement, areas of ranges of the respective particle diameters were determined, and from area ratios thereof, a volume proportion of particle diameters of 45 μm or more relative to the volume of the whole of particles was determined.

With respect to particles of (B1) shown in Table 1, the measurement was performed using a dynamic light scattering particle size distribution analyzer “LB-500”, manufactured by Horiba, Ltd. The measurement was similarly performed using a dynamic light scattering particle size distribution analyzer (“LB-500”, manufactured by Horiba, Ltd., measuring condition: particle diameter basis (volume), relative refractive index: 1.05-0.0000 by using Silicone 6cs (A1) of the component (A) as a dispersion medium.

### (Evaluation of Repellent Sustaining Effect Against Pests)

(I) Preparation of Aedes albopictus

As the Aedes albopictus, imagoes grown by breeding eggs of Aedes albopictus provided from Sumitomo Technoservice Co., Ltd. in a cage under a condition at 27° C. and a relative humidity (RH) of 60% were used.

A transparent plastic pan was filled with water in a depth of about 1 cm, and a filter paper having the purchased eggs laid thereon was put therein, thereby hatching the eggs into pupae. Thereafter, the hatched pupae were fed with a bait (tetramine) for tropical fish as a food for larvae every day. One week later, the pupae were collected with a dropper, transferred into a 20-mL plastic cup, which was then transferred into a net-spread cage. The pupae were fed with 10% by mass of sucrose contained in a 25-mL plastic tube as an adult food. After emergence, males and females were bred in the same cage for 5 days for mating. After 5 days of breeding, adult worms were collected using a fluke tube, the males and the females were visually separated after anesthesia for 5 minutes on ice, and only the females were collected and used for evaluation.

### (II) Measurement of Repellent Effect Sustaining Time

About 100 females of Aedes albopictus were transferred into a plastic cage (30×30×30 cm), and the test was carried out under a condition at 27° C. and a relative humidity (RH) of 60%. A hole having a size of 4×5 cm was provided in a forearm part of a rubber-made glove having a size so as to cover the arm of a subject, and the composition of each of the Examples was uniformly applied at a coverage of 2 mg/cm².

Thereafter, in order to activate the Aedes albopictus, breathing on the plastic cage was performed for 5 seconds. The test was performed in such a manner that the forearm part immediately after applying the composition was put into the plastic cage and exposed with the Aedes albopictus for 2 minutes, to count the number of stays.

At the point of time when the staying was confirmed two times in total, the test was finished, and the exposure for 2 minutes was performed at intervals of 30 minutes until the test was finished. In the case where the staying at the second time was confirmed during the exposure at 30 minutes, the repellent effect sustaining time was judged to be 0 minute, and in the case where the staying at the second time was confirmed during the exposure at 60 minutes, the repellent effect sustaining time was judged to be 30 minutes. The test was performed by three subjects, and an average repellent effect sustaining time was calculated.

(Evaluation of Feeling (Dry Feeling During Applying onto the Skin))

In three expert panelists, 0.02 mL of the composition of each of the Examples was applied onto a circle having a diameter of 3 cm of an inner part of the forearm and spread over one minute under a condition at 25° C. and 57 RH %. A dry feeling during drying and after drying was subjected to sensory evaluation on 5 grades according to the following criteria, and an average score by three persons was expressed in the tables. The evaluation result of Comparative Example 1 was designated as “1”.

5: A sticky feeling is not confirmed at all, and a good dry feeling is provided.

4: Although stickiness is slightly felt, a dry feeling is provided.

3: Although stickiness is felt a little, a dry feeling is rather stronger.

2: Stickiness is felt, and a dry feeling is weak.

1: Stickiness is felt, and a dry feeling is not provided.

### (Evaluation of Long-Term Storage Stability)

80 mL of the composition of each of the Examples was placed in a 100-mL glass bottle, sealed, and then stored at 50° C. for 1 month. Thereafter, the presence or absence of separation of an oil phase in the composition was confirmed through visual inspection and expressed according to the following criteria.

A: Not changed

B: The oil phase coalesces, and some exudation from the emulsion to the surface can be seen.

C: Separation of the oil phase is observed.

### Production Example 1 (Production of Dimethylsilylated Cellulose Particles (B10))

In a 2,000-mL separable flask, cellulose particles (CELLULOBEADS D-30, manufactured by Daito Kasei Kogyo co., Ltd., 30-μm spherical cellulose) were weighed in an amount of 100.00 g in terms of a water-containing mass, and for the purpose of removing the moisture in the cellulose particles, the cellulose particles were vacuum dried by heating at 80° C. until the final moisture value became 1% by mass or less. In the 2,000-mL separable flask containing the dried cellulose particles, 600 mL of super dehydrated N,N-dimethylformamide (manufactured by FUJIFILM Wako Pure Chemical Corporation) was added and stirred at room temperature for 30 minutes, and then, 10.00 g of chlorodimethylsilane (manufactured by Tokyo Chemical Industry Co., Ltd.) was gradually added dropwise. After completion of the dropwise addition, the mixture was subjected to temperature rise on a water bath to 80° C. in terms of an internal temperature and reacted for 4 hours. After completion of the reaction, the reaction mixture was subjected to filtration fractionation, and the filtrate was washed with acetone. Thereafter, the filtrate was vacuum dried by heating at 80° C. for a whole day and night, to obtain dimethylsilylated cellulose particles (B10) as hydrophobic particles having a volume median particle diameter (D50) of 33.4 μm.

### Comparative Production Example 1 (Production of Comparative Dimethylsilylated Cellulose Particles (b4))

Comparative dimethylsilylated cellulose particles (b4) as hydrophobic particles having a volume median particle diameter (D50) of 45.4 μm were obtained by the same method as in Production Example 1, except for using cellulose particles “CELLULOBEADS D-50” (manufactured by Daito Kasei Kogyo co., Ltd., 50-μm spherical cellulose) in place of the CELLULOBEADS D-30 (manufactured by Daito Kasei Kogyo co., Ltd.).

### Examples 1 to 57 and Comparative Examples 1 to 12 (Production and Evaluation of Composition)

Among the respective components shown in Tables 2 to 4 and Tables 6 to 7, the particle component and water were previously blended and mixed by using an apparatus in which a stirring unit of a T.K. ROBOMIX (manufactured by Tokushu Kika Kogyo Co., Ltd.) was replaced by a T.K. homodisper 2.5 type. Then, the component (A) and the component (a), and the thickener in amounts shown in the tables were blended, and oil-in-water type compositions were obtained through a step of stirring at 8,000 rpm for 2 minutes by using an apparatus in which a stirring unit of a T.K. ROBOMIX (manufactured by Tokushu Kika Kogyo Co., Ltd.) was replaced by a T.K. homomixer MARKII 2.5 type. The expressions (A) to (D) listed in the tables are corresponding to the components (A) to (D) in this specification, respectively, and the expressions (a) and (b) are corresponding to the liquid oily component falling outside the scope of the component (A) and the particles falling outside the scope of the component (B), respectively in this specification. In addition, the components used in Tables 2 to 4 are shown in Table 1, and the components used in Tables 6 to 7 are shown in Table 5. The blending amounts shown in the respective tables are the active ingredient amounts (% by mass) of the respective components. The various evaluations were performed by the aforementioned methods by using the obtained compositions. The results are shown in the tables.

TABLE 1

| | | | | Surface | |
|---|---|---|---|---|---|
| | | | | tension | Viscosity |
| | | | | at 25° C. | at 23° C. |
| Trade name | Manufacturer name | Classification | Component name | [mN/m] | [mPa · s] |
| Silicone KF-96A-6cs | Shin-Etsu Chemical | (A1) | Dimethylpolysiloxane | 19.0 | 5 |
| | Co., Ltd. | | | | |
| KSG-16 | Shin-Etsu Chemical | (B1) | (Dimethicone/vinyl | — | — |
| | Co., Ltd. | | dimethicone) crosspolymer | | |
| | | (A1) | Dimethylpolysiloxane | 19.0 | — |
| KSP-105 | Shin-Etsu Chemical | (B2) | (Vinyl dimethicone/ | — | — |
| | Co., Ltd. | | methiconesilsesquioxane) | | |
| | | | crosspolymer | | |
| Resin powder A | — | (B3) | (Lauryl methacrylate/Na | — | — |
| described in [0046] | | | methacrylate) crosspolymer | | |
| to [0047] Production | | | | | |
| Example 1 of | | | | | |
| JP 2006-225311 A | | | | | |
| KSP-100 | Shin-Etsu Chemical | (B4) | (Vinyl dimethicone/ | — | — |
| | Co., Ltd. | | methiconesilsesquioxane) | | |
| | | | crosspolymer | | |
| GANZPEARL SIG-070 | Aica Kogyo Co., Ltd. | (B5) | Polymethyl methacrylate | — | — |
| GANZPEARL | Aica Kogyo Co., Ltd. | (B6) | Polymethyl methacrylate | — | — |
| GMP-0800 | | | | | |
| KSP-101 | Shin-Etsu Chemical | (B7) | (Vinyl dimethicone/ | — | — |
| | Co., Ltd. | | methiconesilsesquioxane) | | |
| | | | crosspolymer | | |
| GANZPEARL GMX-2001 | Aica Kogyo Co., Ltd. | (B8) | Polymethyl methacrylate | — | — |
| KSP-102 | Shin-Etsu Chemical | (B9) | (Vinyl dimethicone/ | — | — |
| | Co., Ltd. | | methiconesilsesquioxane) | | |
| | | | crosspolymer | | |
| Dimethylsilylated | — | (B10) | Dimethylsilylated | — | — |
| cellulose | | | cellulose | | |
| particles obtained | | | | | |
| in Production | | | | | |
| Example 1 | | | | | |
| Fine particle | Tayca | (b1) | Titanium oxide | — | — |
| titanium oxide | Corporation | | | | |
| MT-100TV | | | | | |
| Godd Ball E-90C | Suzuki Yushi | (b2) | Silica | — | — |
| | Kogyo K.K. | | | | |
| Comparative | — | (b3) | Dimethylsilylated | — | — |
| dimethylsilylated | | | cellulose | | |
| cellulose particles | | | | | |
| obtained in | | | | | |
| Comparative | | | | | |
| Production Example 1 | | | | | |

| | | | |
|---|---|---|---|
| Aristoflex HMB | Clariant Iberica | (D1) | (Ammonium acryloyldimethyltaurate/ |
| | Production S.A. | | beheneth-25 methacrylate) crosspolymer |

| | | | | Volume proportion |
|---|---|---|---|---|
| | | | Volume | of particle diameter |
| | | | median | of 45 μm or more |
| | Wetting | | particle | relative to the |
| | tension | Component | diameter | volume of the whole |
| | at 23° C. | composition | (D50) | of particles |
| Trade name | [mN/m] | [% by mass] | [μm] | [%] |
| Silicone KF-96A-6cs | — | 100 | — | — |
| KSG-16 | 40 | 24 | 0.55 | — |
| | — | 76 | — | |
| KSP-105 | 44 | 100 | 1.8 | 0 |
| Resin powder A | 42 | 100 | 3.2 | ≤0.1 |
| described in [0046] | | | | |
| to [0047] Production | | | | |
| Example 1 of | | | | |
| JP 2006-225311 A | | | | |
| KSP-100 | 40 | 100 | 5.6 | 3.3 |
| GANZPEARL SIG-070 | 44 | 100 | 12.5 | ≤0.1 |
| GANZPEARL | 44 | 100 | 9.0 | 0.7 |
| GMP-0800 | | | | |
| KSP-101 | 40 | 100 | 12.9 | 2.6 |
| GANZPEARL GMX-2001 | 46 | 100 | 17.5 | 0.1 |
| KSP-102 | 38 | 100 | 24.9 | 8.0 |
| Dimethylsilylated | 29 | 100 | 33.4 | — |
| cellulose | | | | |
| particles obtained | | | | |
| in Production | | | | |
| Example 1 | | | | |
| Fine particle | 38 | 100 | 0.074 | — |
| titanium oxide | | | | |
| MT-100TV | | | | |
| Godd Ball E-90C | >70 | 100 | 24.1 | — |
| Comparative | 29 | 100 | 45.2 | — |
| dimethylsilylated | | | | |
| cellulose particles | | | | |
| obtained in | | | | |
| Comparative | | | | |
| Production Example 1 | | | | |
| Aristoflex HMB | (Ammonium | 100 | — | — |
| | acryloyldimethyltaurate/ | | | |
| | beheneth-25 | | | |
| | methacrylate) crosspolymer | | | |

TABLE 2

| | | | | Volume | | | | | | | |
|---|---|---|---|---|---|---|---|---|---|---|---|
| | | | | median | | | | | | | |
| | | Surface | Wetting | particle | | | | | | | |
| | | tension | tension | diameter | | | | | | | |
| | | at 25° C. | at 23° C. | (D50) | Example | | | | | | |
| % by mass | | [mN/m] | [mN/m] | [μm] | 1 | 2 | 3 | 4 | 5 | 6 | 7 |
| (A1) | Dimethylpolysiloxane | 19.0 | — | — | 30.00 | 30.00 | 30.00 | 30.00 | 30.00 | 30.00 | 30.00 |
| (B1) | (Dimethicone/vinyl | — | 40 | 0.55 | 5.00 | 0.00 | 0.00 | 0.00 | 0.00 | 0.00 | 0.00 |
| | dimethicone) crosspolymer | | | | | | | | | | |
| (B2) | (Vinyl dimethicone/ | — | 44 | 1.8 | 0.00 | 5.00 | 0.00 | 0.00 | 0.00 | 0.00 | 0.00 |
| | methiconesilsesquioxane) | | | | | | | | | | |
| | crosspolymer | | | | | | | | | | |
| (B3) | (Lauryl methacrylate/Na | — | 42 | 3.2 | 0.00 | 0.00 | 5.00 | 0.00 | 0.00 | 0.00 | 0.00 |
| | methacrylate) crosspolymer | | | | | | | | | | |
| (B4) | (Vinyl dimethicone/ | — | 40 | 5.6 | 0.00 | 0.00 | 0.00 | 5.00 | 0.00 | 0.00 | 0.00 |
| | methiconesilsesquioxane) | | | | | | | | | | |
| | crosspolymer | | | | | | | | | | |
| (B5) | Polymethyl methacrylate | — | 44 | 12.5 | 0.00 | 0.00 | 0.00 | 0.00 | 5.00 | 0.00 | 0.00 |
| (B6) | Polymethyl methacrylate | — | 44 | 9.0 | 0.00 | 0.00 | 0.00 | 0.00 | 0.00 | 5.00 | 0.00 |
| (B7) | (Vinyl dimethicone/ | — | 40 | 12.9 | 0.00 | 0.00 | 0.00 | 0.00 | 0.00 | 0.00 | 5.00 |
| | methiconesilsesquioxane) | | | | | | | | | | |
| | crosspolymer | | | | | | | | | | |
| (B8) | Polymethyl methacrylate | — | 46 | 17.5 | 0.00 | 0.00 | 0.00 | 0.00 | 0.00 | 0.00 | 0.00 |
| (B9) | (Vinyl dimethicone/ | — | 38 | 24.9 | 0.00 | 0.00 | 0.00 | 0.00 | 0.00 | 0.00 | 0.00 |
| | methiconesilsesquioxane) | | | | | | | | | | |
| | crosspolymer | | | | | | | | | | |
| (B10) | Dimethylsilylated cellulose | — | 29 | 33.4 | 0.00 | 0.00 | 0.00 | 0.00 | 0.00 | 0.00 | 0.00 |
| | particles | | | | | | | | | | |
| (b1) | Titanium oxide | — | 38 | 0.074 | 0.00 | 0.00 | 0.00 | 0.00 | 0.00 | 0.00 | 0.00 |
| (b2) | Silica | — | >70 | 24.1 | 0.00 | 0.00 | 0.00 | 0.00 | 0.00 | 0.00 | 0.00 |
| (D1) | (Ammonium acryloyldimethyltaurate/ | | | | 0.50 | 0.50 | 0.50 | 0.50 | 0.50 | 0.50 | 0.50 |
| | beheneth-25 methacrylate) crosspolymer | | | | | | | | | | |
| (C) | Water | | | | 64.50 | 64.50 | 64.50 | 64.50 | 64.50 | 64.50 | 64.50 |
| Total | | | | | 100.0 | 100.0 | 100.0 | 100.0 | 100.0 | 100.0 | 100.0 |
| Total content of particles (% by mass) | | | | | 5.00 | 5.00 | 5.00 | 5.00 | 5.00 | 5.00 | 5.00 |
| volume median particle diameter of particles (D50) [μm] | | | | | 0.6 | 1.8 | 3.2 | 5.6 | 12.5 | 9.0 | 12.9 |
| Content of component (A) (% by mass) | | | | | 30.0 | 30.0 | 30.0 | 30.0 | 30.0 | 30.0 | 30.0 |
| Content of component (B) (% by mass) | | | | | 5.0 | 5.0 | 5.0 | 5.0 | 5.0 | 5.0 | 5.0 |
| Mass ratio (A)/(B) | | | | | 6.0 | 6.0 | 6.0 | 6.0 | 6.0 | 6.0 | 6.0 |
| Mass ratio (A)/(C) | | | | | 0.5 | 0.5 | 0.5 | 0.5 | 0.5 | 0.5 | 0.5 |
| Repellent | Repellent effect immediately after use | | | | Stay: 0 | Stay: 0 | Stay: 0 | Stay: 0 | Stay: 0 | Stay: 0 | Stay: 0 |
| effect | 30 min after use | | | | Stay: 0 | Stay: 0 | Stay: 0 | Stay: 0 | Stay: 0 | Stay: 0 | Stay: 0 |
| sustaining | 60 min after use | | | | Stay: 0 | Stay: 0 | Stay: 0 | Stay: 0 | Stay: 0 | Stay: 0 | Stay: 0 |
| time | 90 min after use | | | | Stay: 0 | Stay: 0 | Stay: 0 | Stay: 0 | Stay: 0 | Stay: 0 | Stay: 0 |
| | 120 min after use | | | | Stay: 0 | Stay: 0 | Stay: 0 | Stay: 0 | Stay: 0 | Stay: 0 | Stay: 0 |
| | 150 min after use | | | | Stay: 0 | Stay: 0 | Stay: 0 | Stay: 0 | Stay: 0 | Stay: 0 | Stay: 0 |
| | 180 min after use | | | | Stay: 0 | Stay: 0 | Stay: 0 | Stay: 0 | Stay 1 | Stay 1 | Stay 1 |
| | 210 min after use | | | | Stay: 0 | Stay 2 | Stay 2 | Stay 2 | Stay 1 | Stay 1 | Stay 1 |
| | 240 min after use | | | | Stay: 1 | | | | | | |
| | 270 min after use | | | | Stay: 1 | | | | | | |
| | 300 min after use | | | | | | | | | | |
| | 330 min after use | | | | | | | | | | |
| | 360 min after use | | | | | | | | | | |
| | 390 min after use | | | | | | | | | | |
| Feeling (dry feeling during applying onto the skin; | | | | | 2.7 | 3.0 | 3.3 | 3.0 | 3.3 | 3.0 | 3.0 |
| 5-grade evaluation) | | | | | | | | | | | |
| Long-term storage stability | | | | | B | B | A | B | B | B | B |
| | | | | Volume | | | | | | | |
| | | | | median | | | | | | | |
| | | Surface | Wetting | particle | | | | | | | |
| | | tension | tension | diameter | | | | | | | |
| | | at 25° C. | at 23° C. | (D50) | Example | | | | | | |
| % by mass | | [mN/m] | [mN/m] | [μm] | 8 | 9 | 10 | 11 | 12 | 13 | 14 |
| (A1) | Dimethylpolysiloxane | 19.0 | — | — | 30.00 | 30.00 | 30.00 | 30.00 | 30.00 | 30.00 | 30.00 |
| (B1) | (Dimethicone/vinyl | — | 40 | 0.55 | 0.00 | 0.00 | 0.00 | 1.00 | 0.00 | 0.00 | 0.00 |
| | dimethicone) crosspolymer | | | | | | | | | | |
| (B2) | (Vinyl dimethicone/ | — | 44 | 1.8 | 0.00 | 0.00 | 0.00 | 0.00 | 0.00 | 0.00 | 0.00 |
| | methiconesilsesquioxane) | | | | | | | | | | |
| | crosspolymer | | | | | | | | | | |
| (B3) | (Lauryl methacrylate/Na | — | 42 | 3.2 | 0.00 | 0.00 | 0.00 | 0.00 | 1.00 | 10.00 | 0.00 |
| | methacrylate) crosspolymer | | | | | | | | | | |
| (B4) | (Vinyl dimethicone/ | — | 40 | 5.6 | 0.00 | 0.00 | 0.00 | 0.00 | 0.00 | 0.00 | 0.00 |
| | methiconesilsesquioxane) | | | | | | | | | | |
| | crosspolymer | | | | | | | | | | |
| (B5) | Polymethyl methacrylate | — | 44 | 12.5 | 0.00 | 0.00 | 0.00 | 0.00 | 0.00 | 0.00 | 1.00 |
| (B6) | Polymethyl methacrylate | — | 44 | 9.0 | 0.00 | 0.00 | 0.00 | 0.00 | 0.00 | 0.00 | 0.00 |
| (B7) | (Vinyl dimethicone/ | — | 40 | 12.9 | 0.00 | 0.00 | 0.00 | 0.00 | 0.00 | 0.00 | 0.00 |
| | methiconesilsesquioxane) | | | | | | | | | | |
| | crosspolymer | | | | | | | | | | |
| (B8) | Polymethyl methacrylate | — | 46 | 17.5 | 5.00 | 0.00 | 0.00 | 0.00 | 0.00 | 0.00 | 0.00 |
| (B9) | (Vinyl dimethicone/ | — | 38 | 24.9 | 0.00 | 5.00 | 0.00 | 0.00 | 0.00 | 0.00 | 0.00 |
| | methiconesilsesquioxane) | | | | | | | | | | |
| | crosspolymer | | | | | | | | | | |
| (B10) | Dimethylsilylated cellulose | — | 29 | 33.4 | 0.00 | 0.00 | 5.00 | 0.00 | 0.00 | 0.00 | 0.00 |
| | particles | | | | | | | | | | |
| (b1) | Titanium oxide | — | 38 | 0.074 | 0.00 | 0.00 | 0.00 | 0.00 | 0.00 | 0.00 | 0.00 |
| (b2) | Silica | — | >70 | 24.1 | 0.00 | 0.00 | 0.00 | 0.00 | 0.00 | 0.00 | 0.00 |

| (D1) | (Ammonium acryloyldimethyltaurate/ | 0.50 | 0.50 | 0.50 | 0.50 | 0.50 | 0.50 | 0.50 |
|---|---|---|---|---|---|---|---|---|
| | beheneth-25 methacrylate) crosspolymer | | | | | | | |
| (C) | Water | 64.50 | 64.50 | 64.50 | 68.50 | 68.50 | 59.50 | 68.50 |
| Total | | 100.0 | 100.0 | 100.0 | 100.0 | 100.0 | 100.0 | 100.0 |
| Total content of particles (% by mass) | | 5.00 | 5.00 | 5.00 | 1.00 | 1.00 | 10.00 | 1.00 |
| volume median particle diameter of particles (D50) [μm] | | 17.5 | 24.9 | 33.4 | 0.6 | 3.2 | 3.2 | 12.5 |
| Content of component (A) (% by mass) | | 30.0 | 30.0 | 30.0 | 30.0 | 30.0 | 30.0 | 30.0 |
| Content of component (B) (% by mass) | | 5.0 | 5.0 | 5.0 | 1.0 | 1.0 | 10.0 | 1.0 |
| Mass ratio (A)/(B) | | 6.0 | 6.0 | 6.0 | 30.0 | 30.0 | 3.0 | 30.0 |
| Mass ratio (A)/(C) | | 0.5 | 0.5 | 0.5 | 0.4 | 0.4 | 0.5 | 0.4 |
| Repellent | Repellent effect immediately after use | Stay: 0 | Stay: 0 | Stay: 0 | Stay: 0 | Stay: 0 | Stay: 0 | Stay: 0 |
| effect | 30 min after use | Stay: 0 | Stay: 0 | Stay: 0 | Stay: 0 | Stay: 0 | Stay: 0 | Stay: 0 |
| sustaining | 60 min after use | Stay: 0 | Stay: 0 | Stay: 0 | Stay: 0 | Stay: 0 | Stay: 0 | Stay: 0 |
| time | 90 min after use | Stay: 0 | Stay: 0 | Stay: 0 | Stay: 0 | Stay: 0 | Stay: 0 | Stay: 0 |
| | 120 min after use | Stay: 0 | Stay: 0 | Stay: 0 | Stay: 1 | Stay: 0 | Stay: 0 | Stay: 1 |
| | 150 min after use | Stay: 0 | Stay: 0 | Stay: 0 | Stay: 1 | Stay: 2 | Stay: 0 | Stay: 1 |
| | 180 min after use | Stay 1 | Stay 1 | Stay: 2 | | | Stay: 0 | |
| | 210 min after use | Stay 1 | Stay 1 | | | | Stay: 0 | |
| | 240 min after use | | | | | | Stay: 0 | |
| | 270 min after use | | | | | | Stay: 0 | |
| | 300 min after use | | | | | | Stay: 0 | |
| | 330 min after use | | | | | | Stay: 2 | |
| | 360 min after use | | | | | | | |
| | 390 min after use | | | | | | | |

| | | | | | | | |
|---|---|---|---|---|---|---|---|
| Feeling (dry feeling during applying onto the skin; | 3.7 | 3.3 | 3.0 | 2.3 | 2.7 | 4.0 | 2.7 |
| 5-grade evaluation) | | | | | | | |
| Long-term storage stability | B | B | B | B | B | B | A |

| | | | | Volume | | | | | | |
|---|---|---|---|---|---|---|---|---|---|---|
| | | | | median | | | | | | |
| | | Surface | Wetting | particle | | | | | | |
| | | tension | tension | diameter | | | | | | |
| | | at 25° C. | at 23° C. | (D50) | Example | | | | | |
| % by mass | | [mN/m] | [mN/m] | [μm] | 15 | 16 | 17 | 18 | 19 | 20 |
| (A1) | Dimethylpolysiloxane | 19.0 | — | — | 30.00 | 30.00 | 30.00 | 30.00 | 30.00 | 30.00 |
| (B1) | (Dimethicone/vinyl | — | 40 | 0.55 | 0.00 | 0.00 | 0.00 | 0.00 | 0.00 | 0.00 |
| | dimethicone) crosspolymer | | | | | | | | | |
| (B2) | (Vinyl dimethicone/ | — | 44 | 1.8 | 0.00 | 0.00 | 0.00 | 0.00 | 0.00 | 0.00 |
| | methiconesilsesquioxane) | | | | | | | | | |
| | crosspolymer | | | | | | | | | |
| (B3) | (Lauryl methacrylate/Na | — | 42 | 3.2 | 0.00 | 0.00 | 0.00 | 0.00 | 0.00 | 0.00 |
| | methacrylate) crosspolymer | | | | | | | | | |
| (B4) | (Vinyl dimethicone/ | — | 40 | 5.6 | 0.00 | 0.00 | 0.00 | 0.00 | 0.00 | 0.00 |
| | methiconesilsesquioxane) | | | | | | | | | |
| | crosspolymer | | | | | | | | | |
| (B5) | Polymethyl methacrylate | — | 44 | 12.5 | 10.00 | 0.00 | 0.00 | 0.00 | 0.00 | 0.00 |
| (B6) | Polymethyl methacrylate | — | 44 | 9.0 | 0.00 | 0.00 | 0.00 | 0.00 | 0.00 | 0.00 |
| (B7) | (Vinyl dimethicone/ | — | 40 | 12.9 | 0.00 | 0.00 | 0.00 | 0.00 | 0.00 | 0.00 |
| | methiconesilsesquioxane) | | | | | | | | | |
| | crosspolymer | | | | | | | | | |
| (B8) | Polymethyl methacrylate | — | 46 | 17.5 | 0.00 | 1.00 | 10.00 | 0.00 | 0.00 | 0.00 |
| (B9) | (Vinyl dimethicone/ | — | 38 | 24.9 | 0.00 | 0.00 | 0.00 | 1.00 | 10.00 | 25.00 |
| | methiconesilsesquioxane) | | | | | | | | | |
| | crosspolymer | | | | | | | | | |
| (B10) | Dimethylsilylated cellulose | — | 29 | 33.4 | 0.00 | 0.00 | 0.00 | 0.00 | 0.00 | 0.00 |
| | particles | | | | | | | | | |
| (b1) | Titanium oxide | — | 38 | 0.074 | 0.00 | 0.00 | 0.00 | 0.00 | 0.00 | 0.00 |
| (b2) | Silica | — | >70 | 24.1 | 0.00 | 0.00 | 0.00 | 0.00 | 0.00 | 0.00 |

| (D1) | (Ammonium acryloyldimethyltaurate/ | 0.50 | 0.50 | 0.50 | 0.50 | 0.50 | 0.50 |
|---|---|---|---|---|---|---|---|
| | beheneth-25 methacrylate) crosspolymer | | | | | | |
| (C) | Water | 59.50 | 68.50 | 59.50 | 68.50 | 59.50 | 44.50 |
| Total | | 100.0 | 100.0 | 100.0 | 100.0 | 100.0 | 100.0 |
| Total content of particles (% by mass) | | 10.00 | 1.00 | 10.00 | 1.00 | 10.00 | 25.00 |
| volume median particle diameter of particles (D50) [μm] | | 12.5 | 17.5 | 17.5 | 24.9 | 24.9 | 24.9 |
| Content of component (A) (% by mass) | | 30.0 | 30.0 | 30.0 | 30.0 | 30.0 | 30.0 |
| Content of component (B) (% by mass) | | 10.0 | 1.0 | 10.0 | 1.0 | 10.0 | 25.0 |
| Mass ratio (A)/(B) | | 3.0 | 30.0 | 3.0 | 30.0 | 3.0 | 1.2 |
| Mass ratio (A)/(C) | | 0.5 | 0.4 | 0.5 | 0.4 | 0.5 | 0.7 |
| Repellent | Repellent effect immediately after use | Stay: 0 | Stay: 0 | Stay: 0 | Stay: 0 | Stay: 0 | Stay: 0 |
| effect | 30 min after use | Stay: 0 | Stay: 0 | Stay: 0 | Stay: 0 | Stay: 0 | Stay: 0 |
| sustaining | 60 min after use | Stay: 0 | Stay: 0 | Stay: 0 | Stay: 0 | Stay: 0 | Stay: 0 |
| time | 90 min after use | Stay: 0 | Stay: 0 | Stay: 0 | Stay: 0 | Stay: 0 | Stay: 0 |
| | 120 min after use | Stay: 0 | Stay: 1 | Stay: 0 | Stay: 1 | Stay: 0 | Stay: 0 |
| | 150 min after use | Stay: 0 | Stay: 1 | Stay: 0 | Stay: 1 | Stay: 0 | Stay: 0 |
| | 180 min after use | Stay: 0 | | Stay: 0 | | Stay: 0 | Stay: 1 |
| | 210 min after use | Stay: 0 | | Stay: 0 | | Stay: 0 | Stay: 1 |
| | 240 min after use | Stay: 0 | | Stay: 0 | | Stay: 0 | |
| | 270 min after use | Stay: 0 | | Stay: 0 | | Stay: 0 | |
| | 300 min after use | Stay: 0 | | Stay: 0 | | Stay: 1 | |
| | 330 min after use | Stay: 0 | | Stay: 0 | | Stay: 1 | |
| | 360 min after use | Stay: 2 | | Stay: 2 | | | |
| | 390 min after use | | | | | | |

| | | | | | | |
|---|---|---|---|---|---|---|
| Feeling (dry feeling during applying onto the skin; | 4.7 | 2.7 | 4.7 | 2.7 | 4.7 | 5.0 |
| 5-grade evaluation) | | | | | | |
| Long-term storage stability | B | B | B | B | B | A |

TABLE 3

| | | | | Volume | | | | | | |
|---|---|---|---|---|---|---|---|---|---|---|
| | | | | median | | | | | | |
| | | Surface | Wetting | particle | | | | | | |
| | | tension | tension | diameter | | | | | | |
| | | at 25° C. | at 23° C. | (D50) | Example | | | | | |
| % by mass | | [mN/m] | [mN/m] | [μm] | 21 | 22 | 23 | 24 | 25 | 26 |
| (A1) | Dimethylpolysiloxane | 19.0 | — | — | 30.00 | 30.00 | 30.00 | 30.00 | 30.00 | 30.00 |
| (B1) | (Dimethicone/vinyl | — | 40 | 0.55 | 0.00 | 0.00 | 0.00 | 2.50 | 5.00 | 7.50 |
| | dimethicone) crosspolymer | | | | | | | | | |
| (B2) | (Vinyl dimethicone/ | — | 44 | 1.8 | 0.00 | 0.00 | 0.00 | 0.00 | 0.00 | 0.00 |
| | methiconesilsesquioxane) | | | | | | | | | |
| | crosspolymer | | | | | | | | | |
| (B3) | (Lauryl methacrylate/Na | — | 42 | 3.2 | 0.00 | 0.00 | 0.00 | 7.50 | 5.00 | 2.50 |
| | methacrylate) crosspolymer | | | | | | | | | |
| (B4) | (Vinyl dimethicone/ | — | 40 | 5.6 | 0.00 | 0.00 | 0.00 | 0.00 | 0.00 | 0.00 |
| | methiconesilsesquioxane) | | | | | | | | | |
| | crosspolymer | | | | | | | | | |
| (B5) | Polymethyl methacrylate | — | 44 | 12.5 | 1.67 | 2.50 | 3.33 | 0.00 | 0.00 | 0.00 |
| (B6) | Polymethyl methacrylate | — | 44 | 9.0 | 0.00 | 0.00 | 0.00 | 0.00 | 0.00 | 0.00 |
| (B7) | (Vinyl dimethicone/ | — | 40 | 12.9 | 0.00 | 0.00 | 0.00 | 0.00 | 0.00 | 0.00 |
| | methiconesilsesquioxane) | | | | | | | | | |
| | crosspolymer | | | | | | | | | |
| (B8) | Polymethyl methacrylate | — | 46 | 17.5 | 0.00 | 0.00 | 0.00 | 0.00 | 0.00 | 0.00 |
| (B9) | (Vinyl dimethicone/ | — | 38 | 24.9 | 3.33 | 2.50 | 1.67 | 0.00 | 0.00 | 0.00 |
| | methiconesilsesquioxane) | | | | | | | | | |
| | crosspolymer | | | | | | | | | |
| (b1) | Titanium oxide | — | 38 | 0.074 | 0.00 | 0.00 | 0.00 | 0.00 | 0.00 | 0.00 |
| (b2) | Silica | — | >70 | 24.1 | 0.00 | 0.00 | 0.00 | 0.00 | 0.00 | 0.00 |

| (D1) | (Ammonium acryloyldimethyltaurate/beheneth-25 | 0.50 | 0.50 | 0.50 | 0.50 | 0.50 | 0.50 |
|---|---|---|---|---|---|---|---|
| | methacrylate) crosspolymer | | | | | | |
| (C) | Water | 64.50 | 64.50 | 64.50 | 59.50 | 59.50 | 59.50 |
| Total | | 100.0 | 100.0 | 100.0 | 100.0 | 100.0 | 100.0 |
| Total content of particles (% by mass) | | 5.00 | 5.00 | 5.00 | 10.00 | 1 | 10.00 |
| volume median particle diameter of particles (D50) [μm] | | 17.6 | 15.8 | 14.1 | 2.2 | 1.6 | 1.0 |
| Content of component (A) (% by mass) | | 30.0 | 30.0 | 30.0 | 30.0 | 30.0 | 30.0 |
| Content of component (B) (% by mass) | | 5.0 | 5.0 | 5.0 | 10.0 | 10.0 | 10.0 |
| Mass ratio (A)/(B) | | 6.0 | 6.0 | 6.0 | 3.0 | 3.0 | 3.0 |
| Mass ratio (A)/(C) | | 0.5 | 0.5 | 0.5 | 0.5 | 0.5 | 0.5 |
| Repellent | Repellent effect immediately after use | Stay: 0 | Stay: 0 | Stay: 0 | Stay: 0 | Stay: 0 | Stay: 0 |
| effect | 30 min after use | Stay: 0 | Stay: 0 | Stay: 0 | Stay: 0 | Stay: 0 | Stay: 0 |
| sustaining | 60 min after use | Stay: 0 | Stay: 0 | Stay: 0 | Stay: 0 | Stay: 0 | Stay: 0 |
| time | 90 min after use | Stay: 0 | Stay: 0 | Stay: 0 | Stay: 0 | Stay: 0 | Stay: 0 |
| | 120 min after use | Stay: 0 | Stay: 0 | Stay: 0 | Stay: 0 | Stay: 0 | Stay: 1 |
| | 150 min after use | Stay: 0 | Stay: 0 | Stay: 0 | Stay: 0 | Stay: 0 | Stay: 1 |
| | 180 min after use | Stay: 0 | Stay: 0 | Stay: 0 | Stay: 0 | Stay: 0 | |
| | 210 min after use | Stay: 2 | Stay: 2 | Stay: 2 | Stay: 0 | Stay: 1 | |
| | 240 min after use | | | | Stay: 0 | Stay: 1 | |
| | 270 min after use | | | | Stay: 0 | | |
| | 300 min after use | | | | Stay: 0 | | |
| | 330 min after use | | | | Stay: 0 | | |
| | 360 min after use | | | | Stay: 2 | | |
| | 390 min after use | | | | | | |

| | | | | | | |
|---|---|---|---|---|---|---|
| Feeling (dry feeling during applying onto the skin; | 3.7 | 3.7 | 3.7 | 3.7 | 3.0 | 2.7 |
| 5-grade evaluation) | | | | | | |
| Long-term storage stability | B | B | B | A | A | B |

| | | | | Volume | | | | | |
|---|---|---|---|---|---|---|---|---|---|
| | | | | median | | | | | |
| | | Surface | Wetting | particle | | | | | |
| | | tension | tension | diameter | | | | | |
| | | at 25° C. | at 23° C. | (D50) | Example | | | | |
| % by mass | | [mN/m] | [mN/m] | [μm] | 27 | 28 | 29 | 30 | 31 |
| (A1) | Dimethylpolysiloxane | 19.0 | — | — | 30.00 | 30.00 | 30.00 | 30.00 | 30.00 |
| (B1) | (Dimethicone/vinyl | — | 40 | 0.55 | 2.50 | 5.00 | 7.50 | 2.50 | 5.00 |
| | dimethicone) crosspolymer | | | | | | | | |
| (B2) | (Vinyl dimethicone/ | — | 44 | 1.8 | 0.00 | 0.00 | 0.00 | 0.00 | 0.00 |
| | methiconesilsesquioxane) | | | | | | | | |
| | crosspolymer | | | | | | | | |
| (B3) | (Lauryl methacrylate/Na | — | 42 | 3.2 | 5.00 | 2.50 | 0.00 | 2.50 | 0.00 |
| | methacrylate) crosspolymer | | | | | | | | |
| (B4) | (Vinyl dimethicone/ | — | 40 | 5.6 | 0.00 | 0.00 | 0.00 | 0.00 | 0.00 |
| | methiconesilsesquioxane) | | | | | | | | |
| | crosspolymer | | | | | | | | |
| (B5) | Polymethyl methacrylate | — | 44 | 12.5 | 0.83 | 0.83 | 0.83 | 1.67 | 1.67 |
| (B6) | Polymethyl methacrylate | — | 44 | 9.0 | 0.00 | 0.00 | 0.00 | 0.00 | 0.00 |
| (B7) | (Vinyl dimethicone/ | — | 40 | 12.9 | 0.00 | 0.00 | 0.00 | 0.00 | 0.00 |
| | methiconesilsesquioxane) | | | | | | | | |
| | crosspolymer | | | | | | | | |
| (B8) | Polymethyl methacrylate | — | 46 | 17.5 | 0.00 | 0.00 | 0.00 | 0.00 | 0.00 |
| (B9) | (Vinyl dimethicone/ | — | 38 | 24.9 | 1.67 | 1.67 | 1.67 | 3.33 | 3.33 |
| | methiconesilsesquioxane) | | | | | | | | |
| | crosspolymer | | | | | | | | |
| (b1) | Titanium oxide | — | 38 | 0.074 | 0.00 | 0.00 | 0.00 | 0.00 | 0.00 |
| (b2) | Silica | — | >70 | 24.1 | 0.00 | 0.00 | 0.00 | 0.00 | 0.00 |

| (D1) | (Ammonium acryloyldimethyltaurate/beheneth-25 | 0.50 | 0.50 | 0.50 | 0.50 | 0.50 |
|---|---|---|---|---|---|---|
| | methacrylate) crosspolymer | | | | | |
| (C) | Water | 59.50 | 59.50 | 59.50 | 59.50 | 59.50 |
| Total | | 100.0 | 100.0 | 100.0 | 100.0 | 100.0 |
| Total content of particles (% by mass) | | 10.00 | 10.00 | 10.00 | 10.00 | 10.00 |
| volume median particle diameter of particles (D50) [μm] | | 5.9 | 5.3 | 4.8 | 9.6 | 9.0 |
| Content of component (A) (% by mass) | | 30.0 | 30.0 | 30.0 | 30.0 | 30.0 |
| Content of component (B) (% by mass) | | 10.0 | 10.0 | 10.0 | 10.0 | 10.0 |
| Mass ratio (A)/(B) | | 3.0 | 3.0 | 3.0 | 3.0 | 3.0 |
| Mass ratio (A)/(C) | | 0.5 | 0.5 | 0.5 | 0.5 | 0.5 |
| Repellent | Repellent effect immediately after use | Stay: 0 | Stay: 0 | Stay: 0 | Stay: 0 | Stay: 0 |
| effect | 30 min after use | Stay: 0 | Stay: 0 | Stay: 0 | Stay: 0 | Stay: 0 |
| sustaining | 60 min after use | Stay: 0 | Stay: 0 | Stay: 0 | Stay: 0 | Stay: 0 |
| time | 90 min after use | Stay: 0 | Stay: 0 | Stay: 0 | Stay: 0 | Stay: 0 |
| | 120 min after use | Stay: 0 | Stay: 0 | Stay: 1 | Stay: 0 | Stay: 1 |
| | 150 min after use | Stay: 0 | Stay: 0 | Stay: 1 | Stay: 0 | Stay: 1 |
| | 180 min after use | Stay: 0 | Stay: 0 | | Stay: 0 | |
| | 210 min after use | Stay: 0 | Stay: 1 | | Stay: 0 | |
| | 240 min after use | Stay: 0 | Stay: 1 | | Stay: 2 | |
| | 270 min after use | Stay: 0 | | | | |
| | 300 min after use | Stay: 0 | | | | |
| | 330 min after use | Stay: 1 | | | | |
| | 360 min after use | Stay: 1 | | | | |
| | 390 min after use | | | | | |

| | | | | | |
|---|---|---|---|---|---|
| Feeling (dry feeling during applying onto the skin; | 4.0 | 3.7 | 3.0 | 4.3 | 3.7 |
| 5-grade evaluation) | | | | | |
| Long-term storage stability | B | B | B | B | B |

| | | | | Volume | | | | | |
|---|---|---|---|---|---|---|---|---|---|
| | | | | median | | | | | |
| | | Surface | Wetting | particle | | | | | |
| | | tension | tension | diameter | | | | | |
| | | at 25° C. | at 23° C. | (D50) | Example | | | | |
| % by mass | | [mN/m] | [mN/m] | [μm] | 32 | 33 | 34 | 35 | 36 |
| (A1) | Dimethylpolysiloxane | 19.0 | — | — | 30.00 | 30.00 | 30.00 | 30.00 | 30.00 |
| (B1) | (Dimethicone/vinyl | — | 40 | 0.55 | 2.50 | 0.00 | 0.00 | 0.00 | 0.00 |
| | dimethicone) crosspolymer | | | | | | | | |
| (B2) | (Vinyl dimethicone/ | — | 44 | 1.8 | 0.00 | 0.00 | 0.00 | 0.00 | 0.00 |
| | methiconesilsesquioxane) | | | | | | | | |
| | crosspolymer | | | | | | | | |
| (B3) | (Lauryl methacrylate/Na | — | 42 | 3.2 | 0.00 | 0.00 | 5.00 | 7.50 | 2.50 |
| | methacrylate) crosspolymer | | | | | | | | |
| (B4) | (Vinyl dimethicone/ | — | 40 | 5.6 | 0.00 | 0.00 | 0.00 | 0.00 | 0.00 |
| | methiconesilsesquioxane) | | | | | | | | |
| | crosspolymer | | | | | | | | |
| (B5) | Polymethyl methacrylate | — | 44 | 12.5 | 2.50 | 3.33 | 1.67 | 0.83 | 5.00 |
| (B6) | Polymethyl methacrylate | — | 44 | 9.0 | 0.00 | 0.00 | 0.00 | 0.00 | 0.00 |
| (B7) | (Vinyl dimethicone/ | — | 40 | 12.9 | 0.00 | 0.00 | 0.00 | 0.00 | 0.00 |
| | methiconesilsesquioxane) | | | | | | | | |
| | crosspolymer | | | | | | | | |
| (B8) | Polymethyl methacrylate | — | 46 | 17.5 | 0.00 | 0.00 | 0.00 | 0.00 | 0.00 |
| (B9) | (Vinyl dimethicone/ | — | 38 | 24.9 | 5.00 | 6.67 | 3.33 | 1.67 | 2.50 |
| | methiconesilsesquioxane) | | | | | | | | |
| | crosspolymer | | | | | | | | |
| (b1) | Titanium oxide | — | 38 | 0.074 | 0.00 | 0.00 | 0.00 | 0.00 | 0.00 |
| (b2) | Silica | — | >70 | 24.1 | 0.00 | 0.00 | 0.00 | 0.00 | 0.00 |

| (D1) | (Ammonium acryloyldimethyltaurate/beheneth-25 | 0.50 | 0.50 | 0.50 | 0.50 | 0.50 |
|---|---|---|---|---|---|---|
| | methacrylate) crosspolymer | | | | | |
| (C) | Water | 59.50 | 59.50 | 59.50 | 59.50 | 59.50 |
| Total | | 100.0 | 100.0 | 100.0 | 100.0 | 100.0 |
| Total content of particles (% by mass) | | 10.00 | 10.00 | 10.00 | 10.00 | 10.00 |
| volume median particle diameter of particles (D50) [μm] | | 13.3 | 17.6 | 10.2 | 6.4 | 11.3 |
| Content of component (A) (% by mass) | | 30.0 | 30.0 | 30.0 | 30.0 | 30.0 |
| Content of component (B) (% by mass) | | 10.0 | 10.0 | 10.0 | 10.0 | 10.0 |
| Mass ratio (A)/(B) | | 3.0 | 3.0 | 3.0 | 3.0 | 3.0 |
| Mass ratio (A)/(C) | | 0.5 | 0.5 | 0.5 | 0.5 | 0.5 |
| Repellent | Repellent effect immediately after use | Stay: 0 | Stay: 0 | Stay: 0 | Stay: 0 | Stay: 0 |
| effect | 30 min after use | Stay: 0 | Stay: 0 | Stay: 0 | Stay: 0 | Stay: 0 |
| sustaining | 60 min after use | Stay: 0 | Stay: 0 | Stay: 0 | Stay: 0 | Stay: 0 |
| time | 90 min after use | Stay: 0 | Stay: 0 | Stay: 0 | Stay: 0 | Stay: 0 |
| | 120 min after use | Stay: 0 | Stay: 0 | Stay: 0 | Stay: 0 | Stay: 0 |
| | 150 min after use | Stay: 0 | Stay: 0 | Stay: 0 | Stay: 0 | Stay: 0 |
| | 180 min after use | Stay: 0 | Stay: 0 | Stay: 0 | Stay: 0 | Stay: 0 |
| | 210 min after use | Stay: 0 | Stay: 0 | Stay: 0 | Stay: 0 | Stay: 0 |
| | 240 min after use | Stay: 2 | Stay: 0 | Stay: 0 | Stay: 0 | Stay: 0 |
| | 270 min after use | | Stay: 0 | Stay: 0 | Stay: 0 | Stay: 0 |
| | 300 min after use | | Stay: 0 | Stay: 0 | Stay: 0 | Stay: 0 |
| | 330 min after use | | Stay: 1 | Stay: 0 | Stay: 0 | Stay: 1 |
| | 360 min after use | | Stay: 1 | Stay: 0 | Stay: 2 | Stay: 1 |
| | 390 min after use | | | Stay: 2 | | |

| | | | | | |
|---|---|---|---|---|---|
| Feeling (dry feeling during applying onto the skin; | 4.7 | 5.0 | 4.7 | 4.3 | 5.0 |
| 5-grade evaluation) | | | | | |
| Long-term storage stability | B | B | B | B | B |

TABLE 4

| | | | | Volume | | | | | | |
|---|---|---|---|---|---|---|---|---|---|---|
| | | | | median | | | | | | |
| | | Surface | Wetting | particle | | | | | | |
| | | tension at | tension at | diameter | | | | | | |
| | | 25° C. | 23° C. | (D50) | Comparative Example | | | | | |
| % by mass | | [mN/m] | [mN/m] | [μm] | 1 | 2 | 3 | 4 | 5 | 6 |
| (A1) | Dimethylpolysiloxane | 19.0 | — | — | 30.00 | 30.00 | 30.00 | 30.00 | 30.00 | 30.00 |
| (B1) | (Dimethicone/vinyl | — | 40 | 0.55 | 0.00 | 0.00 | 0.00 | 0.00 | 0.00 | 0.00 |
| | dimethicone) crosspolymer | | | | | | | | | |
| (B2) | (Vinyl dimethicone/ | — | 44 | 1.8 | 0.00 | 0.00 | 0.00 | 0.00 | 0.00 | 0.00 |
| | methiconesilsesquioxane) | | | | | | | | | |
| | crosspolymer | | | | | | | | | |
| (B3) | (Lauryl methacrylate/Na | — | 42 | 3.2 | 0.00 | 0.00 | 0.00 | 0.00 | 0.00 | 0.00 |
| | methacrylate) crosspolymer | | | | | | | | | |
| (B4) | (Vinyl dimethicone/ | — | 40 | 5.6 | 0.00 | 0.00 | 0.00 | 0.00 | 0.00 | 0.00 |
| | methiconesilsesquioxane) | | | | | | | | | |
| | crosspolymer | | | | | | | | | |
| (B5) | Polymethyl methacrylate | — | 44 | 12.5 | 0.00 | 0.00 | 0.00 | 0.00 | 0.00 | 0.00 |
| (B6) | Polymethyl methacrylate | — | 44 | 9.0 | 0.00 | 0.00 | 0.00 | 0.00 | 0.00 | 0.00 |
| (B7) | (Vinyl dimethicone/ | — | 40 | 12.9 | 0.00 | 0.00 | 0.00 | 0.00 | 0.00 | 0.00 |
| | methiconesilsesquioxane) | | | | | | | | | |
| | crosspolymer | | | | | | | | | |
| (B8) | Polymethyl methacrylate | — | 46 | 17.5 | 0.00 | 0.00 | 0.00 | 0.00 | 0.00 | 0.00 |
| (B9) | (Vinyl dimethicone/ | — | 38 | 24.9 | 0.00 | 0.10 | 0.00 | 0.00 | 0.00 | 40.00 |
| | methiconesilsesquioxane) | | | | | | | | | |
| | crosspolymer | | | | | | | | | |
| (b1) | Titanium oxide | — | 38 | 0.074 | 0.00 | 0.00 | 5.00 | 0.00 | 0.00 | 0.00 |
| (b2) | Silica | — | >70 | 24.1 | 0.00 | 0.00 | 0.00 | 5.00 | 0.00 | 0.00 |
| (b3) | Dimethylsilylated cellulose | — | 29 | 45.2 | 0.00 | 0.00 | 0.00 | 0.00 | 5.00 | 0.00 |

| (D1) | (Ammonium acryloyldimethyltaurate/ | 0.50 | 0.50 | 0.50 | 0.50 | 0.50 | 0.50 |
|---|---|---|---|---|---|---|---|
| | beheneth-25 methacrylate) crosspolymer | | | | | | |
| (C) | Water | 69.50 | 69.40 | 64.50 | 64.50 | 64.50 | 29.50 |
| Total | | 100.0 | 100.0 | 100.0 | 100.0 | 100.0 | 100.0 |
| Total content of particles (% by mass) | | 0.00 | 0.10 | 5.00 | 5.00 | 5.00 | 40.00 |
| volume median particle diameter of particles (D50) [μm] | | 0 | 24.9 | 0.074 | 24.1 | 45.2 | 24.9 |
| Content of component (A) (% by mass) | | 30.00 | 30.00 | 30.00 | 30.00 | 30.00 | 30.00 |
| Content of component (B) (% by mass) | | 0.00 | 0.10 | 0.00 | 0.00 | 0.00 | 40.00 |
| Mass ratio (A)/(B) | | — | 300.0 | — | — | — | 0.8 |
| Mass ratio (A)/(C) | | 0.4 | 0.4 | 0.5 | 0.5 | 0.5 | 1.0 |
| Repellent | Repellent effect immediately after use | Stay: 0 | Stay: 0 | Stay: 0 | Stay: 0 | Stay: 0 | Stay: 0 |
| effect | 30 min after use | Stay: 0 | Stay: 0 | Stay: 0 | Stay: 0 | Stay: 0 | Stay: 0 |
| sustaining | 60 min after use | Stay: 0 | Stay: 0 | Stay: 0 | Stay: 0 | Stay: 0 | Stay: 0 |
| time | 90 min after use | Stay: 0 | Stay: 0 | Stay: 0 | Stay: 0 | Stay: 0 | Stay: 2 |
| | 120 min after use | Stay: 2 | Stay: 2 | Stay: 0 | Stay: 0 | Stay: 0 | |
| | 150 min after use | | | Stay: 0 | Stay: 0 | Stay: 0 | |
| | 180 min after use | | | Stay: 0 | Stay: 0 | Stay: 2 | |
| | 210 min after use | | | Stay: 0 | Stay: 0 | | |
| | 240 min after use | | | Stay: 1 | Stay: 1 | | |
| | 270 min after use | | | Stay: 1 | Stay: 1 | | |
| | 300 min after use | | | | | | |
| | 330 min after use | | | | | | |
| | 360 min after use | | | | | | |
| | 390 min after use | | | | | | |

| | | | | | | |
|---|---|---|---|---|---|---|
| Feeling (dry feeling during applying onto the skin; | 1.0 | 1.3 | 1.0 | 1.0 | 1.0 | 5.0 |
| 5-grade evaluation) | | | | | | |
| Long-term storage stability | B | A | C | B | B | C |

TABLE 5

| | | | | | | | | Volume |
|---|---|---|---|---|---|---|---|---|
| | | | | | | | | median |
| | | | | Surface | | Wetting | | particle |
| | | | | tension | Viscosity | tension | Component | diameter |
| | | Classifi- | | at 25° C. | at 23° C. | at 23° C. | composition | (D50) |
| Trade name | Manufacturer name | cation | Component name | [mN/m] | [mPa · s] | [mN/m] | [% by mass] | [μm] |
| Silicone KF-96A- | Shin-Etsu Chemical | (A1) | Dimethylpolysiloxane | 19.0 | 5 | — | 100 | — |
| 6cs | Co., Ltd. | | | | | | | |
| Silicone KF-96A- | Shin-Etsu Chemical | (A2) | Dimethylpolysiloxane | 21.0 | 10 | — | 100 | — |
| 10cs | Co., Ltd. | | | | | | | |
| Silicone KF-96A- | Shin-Etsu Chemical | (A3) | Dimethylpolysiloxane | 21.0 | 60 | — | 100 | — |
| 50cs | Co., Ltd. | | | | | | | |
| Silicone KF-96A- | Shin-Etsu Chemical | (A4) | Dimethylpolysiloxane | 21.0 | 210 | — | 100 | — |
| 200cs | Co., Ltd. | | | | | | | |
| Exceparl IPM | Kao Corporation | (A5) | Isopropyl myristate | 28.0 | 5 | — | 100 | — |
| Nikkol Squalane | Nippon Surfactant | (A6) | Squalane | 30.0 | 42 | — | 100 | — |
| | Industries Co., Ltd. | | | | | | | |
| ASE-166K | Kao Corporation | (A7) | Cetyl-1,3-dimethylbutyl | 28.0 | 7 | — | 100 | — |
| | | | ether | | | | | |
| Polypropylene | FUJIFILM Wako Pure | (A8) | Polypropylene glycol/ | 34.0 | 160 | — | 100 | — |
| glycol | Chemical Corporation | | 1,3-butylene glycol | | | | | |
| 1,3-Butylene | | | (mixture having a mass | | | | | |
| glycol | | | ratio of 3/7) | | | | | |
| Silicone KF-96A- | Shin-Etsu Chemical | (a1) | Dimethylpolysiloxane | 21.0 | 560 | — | 100 | — |
| 500cs | Co., Ltd. | | | | | | | |
| Glycerin | FUJIFILM Wako Pure | (a2) | Glycerin | 65.0 | 950 | — | 100 | — |
| | Chemical Corporation | | | | | | | |
| Silicone KF-96A- | Shin-Etsu Chemical | (a3) | Dimethylpolysiloxane | 21.0 | 4500 | — | 100 | — |
| 5000cs | Co., Ltd. | | | | | | | |
| 1,3-Propanediol | FUJIFILM Wako Pure | (a4) | 1,3-Propanediol | 48.0 | 53 | — | 100 | — |
| | Chemical Corporation | | | | | | | |
| Resin powder A | — | (B3) | (Lauryl methacrylate/ | — | — | 42 | 100 | 3.2 |
| described in | | | Na methacrylate) | | | | | |
| [0046] to [0047] | | | crosspolymer | | | | | |
| Production | | | | | | | | |
| Example 1 of JP | | | | | | | | |
| 2006-225311 A | | | | | | | | |
| GANZPEARL | Aica Kogyo Co., Ltd. | (B5) | Polymethyl methacrylate | — | — | 44 | 100 | 12.5 |
| SIG-070 | | | | | | | | |
| KSP-102 | Shin-Etsu Chemical | (B9) | (Vinyl dimethicone/ | — | — | 38 | 100 | 24.9 |
| | Co., Ltd. | | methiconesilsesquioxane) | | | | | |
| | | | crosspolymer | | | | | |

| | | | | | |
|---|---|---|---|---|---|
| Aristoflex HMB | Clariant Iberica | (D1) | (Ammonium acryloyldimethyltaurate/ | 100 | — |
| | Production S.A. | | beheneth-25 methacrylate) crosspolymer | | |

TABLE 6

| | | | | | Volume | | | | | | |
|---|---|---|---|---|---|---|---|---|---|---|---|
| | | | | | median | | | | | | |
| | | Surface | | Wetting | particle | | | | | | |
| | | tension at | Viscosity | tension | diameter | | | | | | |
| | | 25° C. | at 23° C. | at 23° C. | (D50) | Example | | | | | |
| % by mass | | [mN/m] | [mPa · s] | [mN/m] | [μm] | 37 | 38 | 39 | 40 | 41 | 42 |
| (A1) | Dimethylpolysiloxane | 19.0 | 5 | — | — | 30.00 | 15.00 | 45.00 | 0.00 | 0.00 | 0.00 |
| (A2) | Dimethylpolysiloxane | 21.0 | 10 | — | — | 0.00 | 0.00 | 0.00 | 0.00 | 0.00 | 0.00 |
| (A3) | Dimethylpolysiloxane | 21.0 | 60 | — | — | 0.00 | 0.00 | 0.00 | 15.00 | 30.00 | 45.00 |
| (A4) | Dimethylpolysiloxane | 21.0 | 210 | — | — | 0.00 | 0.00 | 0.00 | 0.00 | 0.00 | 0.00 |
| (A5) | Isopropyl myristate | 28.0 | 5 | — | — | 0.00 | 0.00 | 0.00 | 0.00 | 0.00 | 0.00 |
| (A6) | Squalane | 30.0 | 42 | — | — | 0.00 | 0.00 | 0.00 | 0.00 | 0.00 | 0.00 |
| (A7) | Cetyl-1,3-dimethyl- | 28.0 | 7 | — | — | 0.00 | 0.00 | 0.00 | 0.00 | 0.00 | 0.00 |
| | phenyl ether | | | | | | | | | | |
| (A8) | Polypropylene glycol/ | 34.0 | 160 | — | — | 0.00 | 0.00 | 0.00 | 0.00 | 0.00 | 0.00 |
| | 1,3-butylene glycol | | | | | | | | | | |
| | (mixture having a | | | | | | | | | | |
| | mass ratio of 3/7) | | | | | | | | | | |
| (a1) | Dimethylpolysiloxane | 21.0 | 560 | — | — | 0.00 | 0.00 | 0.00 | 0.00 | 0.00 | 0.00 |
| (a2) | Glycerin | 65.0 | 950 | — | — | 0.00 | 0.00 | 0.00 | 0.00 | 0.00 | 0.00 |
| (a3) | Dimethylpolysiloxane | 21.0 | 4500 | — | — | 0.00 | 0.00 | 0.00 | 0.00 | 0.00 | 0.00 |
| (a4) | 1,3-Propanediol | 48.0 | 53 | — | — | 0.00 | 0.00 | 0.00 | 0.00 | 0.00 | 0.00 |
| (B3) | (Lauryl methacrylate/ | — | — | 42 | 3.2 | 5.00 | 5.00 | 5.00 | 5.00 | 5.00 | 5.00 |
| | Na methacrylate) | | | | | | | | | | |
| | crosspolymer | | | | | | | | | | |
| (B5) | Polymethyl methacrylate | — | — | 44 | 12.5 | 1.67 | 1.67 | 1.67 | 1.67 | 1.67 | 1.67 |
| (B9) | (Vinyl dimethicone/ | — | — | 38 | 24.9 | 3.33 | 3.33 | 3.33 | 3.33 | 3.33 | 3.33 |
| | methiconesilsesquioxane) | | | | | | | | | | |
| | crosspolymer | | | | | | | | | | |

| (D1) | (Ammonium acryloyldimethyltaurate/ | 0.50 | 0.50 | 0.50 | 0.50 | 0.50 | 0.50 |
|---|---|---|---|---|---|---|---|
| | beheneth-25 methacrylate) crosspolymer | | | | | | |
| (C) | Water | 59.50 | 74.50 | 44.50 | 74.50 | 59.50 | 44.50 |
| Total | | 100.0 | 100.0 | 100.0 | 100.0 | 100.0 | 100.0 |
| Total content of particles (% by mass) | | 10.0 | 10.0 | 10.0 | 10.0 | 10.0 | 10.0 |
| volume median particle diameter of particles (D50) [μm] | | 10.2 | 10.2 | 10.2 | 10.2 | 10.2 | 10.2 |
| Content of component (A) (% by mass) | | 30.0 | 15.0 | 45.0 | 15.0 | 30.0 | 45.0 |
| Content of component (B) (% by mass) | | 10.0 | 10.0 | 10.0 | 10.0 | 10.0 | 10.0 |
| Mass ratio (A)/(B) | | 3.0 | 1.5 | 4.5 | 1.5 | 3.0 | 4.5 |
| Mass ratio (A)/(C) | | 0.5 | 0.2 | 1.0 | 0.2 | 0.5 | 1.0 |
| Repellent | Repellent effect immediately after use | Stay: 0 | Stay: 0 | Stay: 0 | Stay: 0 | Stay: 0 | Stay: 0 |
| effect | 30 min after use | Stay: 0 | Stay: 0 | Stay: 0 | Stay: 0 | Stay: 0 | Stay: 0 |
| sustaining | 60 min after use | Stay: 0 | Stay: 0 | Stay: 0 | Stay: 0 | Stay: 0 | Stay: 0 |
| time | 90 min after use | Stay: 0 | Stay: 0 | Stay: 0 | Stay: 0 | Stay: 0 | Stay: 0 |
| | 120 min after use | Stay: 0 | Stay: 0 | Stay: 0 | Stay: 0 | Stay: 0 | Stay: 0 |
| | 150 min after use | Stay: 0 | Stay: 0 | Stay: 0 | Stay: 0 | Stay: 0 | Stay: 0 |
| | 180 min after use | Stay: 0 | Stay: 2 | Stay: 0 | Stay: 2 | Stay: 0 | Stay: 0 |
| | 210 min after use | Stay: 0 | | Stay: 0 | | Stay: 0 | Stay: 0 |
| | 240 min after use | Stay: 0 | | Stay: 0 | | Stay: 0 | Stay: 0 |
| | 270 min after use | Stay: 0 | | Stay: 0 | | Stay: 0 | Stay: 0 |
| | 300 min after use | Stay: 0 | | Stay: 0 | | Stay: 0 | Stay: 0 |
| | 330 min after use | Stay: 0 | | Stay: 0 | | Stay: 0 | Stay: 0 |
| | 360 min after use | Stay: 0 | | Stay: 0 | | Stay: 2 | Stay: 0 |
| | 390 min after use | Stay: 2 | | Stay: 0 | | | Stay: 0 |
| | 420 min after use | | | Stay: 0 | | | Stay: 0 |
| | 450 min after use | | | Stay: 1 | | | Stay: 2 |
| | 480 nm after use | | | Stay: 1 | | | |

| | | | | | | |
|---|---|---|---|---|---|---|
| Feeling (dry feeling during applying onto the skin; 5-grade evaluation) | 4.7 | 5.0 | 4.0 | 4.2 | 3.9 | 3.2 |
| Long-term storage stability | B | B | B | B | B | B |

| | | | | | Volume | | | | | |
|---|---|---|---|---|---|---|---|---|---|---|
| | | | | | median | | | | | |
| | | Surface | | Wetting | particle | | | | | |
| | | tension at | Viscosity | tension | diameter | | | | | |
| | | 25° C. | at 23° C. | at 23° C. | (D50) | Example | | | | |
| % by mass | | [mN/m] | [mPa · s] | [mN/m] | [μm] | 43 | 44 | 45 | 46 | 47 |
| (A1) | Dimethylpolysiloxane | 19.0 | 5 | — | — | 0.00 | 0.00 | 0.00 | 0.00 | 0.00 |
| (A2) | Dimethylpolysiloxane | 21.0 | 10 | — | — | 0.00 | 0.00 | 0.00 | 0.00 | 0.00 |
| (A3) | Dimethylpolysiloxane | 21.0 | 60 | — | — | 0.00 | 0.00 | 0.00 | 0.00 | 0.00 |
| (A4) | Dimethylpolysiloxane | 21.0 | 210 | — | — | 15.00 | 30.00 | 45.00 | 0.00 | 0.00 |
| (A5) | Isopropyl myristate | 28.0 | 5 | — | — | 0.00 | 0.00 | 0.00 | 15.00 | 30.00 |
| (A6) | Squalane | 30.0 | 42 | — | — | 0.00 | 0.00 | 0.00 | 0.00 | 0.00 |
| (A7) | Cetyl-1,3-dimethyl- | 28.0 | 7 | — | — | 0.00 | 0.00 | 0.00 | 0.00 | 0.00 |
| | phenyl ether | | | | | | | | | |
| (A8) | Polypropylene glycol/ | 34.0 | 160 | — | — | 0.00 | 0.00 | 0.00 | 0.00 | 0.00 |
| | 1,3-butylene glycol | | | | | | | | | |
| | (mixture having a | | | | | | | | | |
| | mass ratio of 3/7) | | | | | | | | | |
| (a1) | Dimethylpolysiloxane | 21.0 | 560 | — | — | 0.00 | 0.00 | 0.00 | 0.00 | 0.00 |
| (a2) | Glycerin | 65.0 | 950 | — | — | 0.00 | 0.00 | 0.00 | 0.00 | 0.00 |
| (a3) | Dimethylpolysiloxane | 21.0 | 4500 | — | — | 0.00 | 0.00 | 0.00 | 0.00 | 0.00 |
| (a4) | 1,3-Propanediol | 48.0 | 53 | — | — | 0.00 | 0.00 | 0.00 | 0.00 | 0.00 |
| (B3) | (Lauryl methacrylate/ | — | — | 42 | 3.2 | 5.00 | 5.00 | 5.00 | 5.00 | 5.00 |
| | Na methacrylate) | | | | | | | | | |
| | crosspolymer | | | | | | | | | |
| (B5) | Polymethyl methacrylate | — | — | 44 | 12.5 | 1.67 | 1.67 | 1.67 | 1.67 | 1.67 |
| (B9) | (Vinyl dimethicone/ | — | — | 38 | 24.9 | 3.33 | 3.33 | 3.33 | 3.33 | 3.33 |
| | methiconesilsesquioxane) | | | | | | | | | |
| | crosspolymer | | | | | | | | | |

| (D1) | (Ammonium acryloyldimethyltaurate/ | 0.50 | 0.50 | 0.50 | 0.50 | 0.50 |
|---|---|---|---|---|---|---|
| | beheneth-25 methacrylate) crosspolymer | | | | | |
| (C) | Water | 74.50 | 59.50 | 44.50 | 74.50 | 59.50 |
| Total | | 100.0 | 100.0 | 100.0 | 100.0 | 100.0 |
| Total content of particles (% by mass) | | 10.0 | 10.0 | 10.0 | 10.0 | 10.0 |
| volume median particle diameter of particles (D50) [μm] | | 10.2 | 10.2 | 10.2 | 10.2 | 10.2 |
| Content of component (A) (% by mass) | | 15.0 | 30.0 | 45.0 | 15.0 | 30.0 |
| Content of component (B) (% by mass) | | 10.0 | 10.0 | 10.0 | 10.0 | 10.0 |
| Mass ratio (A)/(B) | | 1.5 | 3.0 | 4.5 | 1.5 | 3.0 |
| Mass ratio (A)/(C) | | 0.2 | 0.5 | 1.0 | 0.2 | 0.5 |
| Repellent | Repellent effect immediately after use | Stay: 0 | Stay: 0 | Stay: 0 | Stay: 0 | Stay: 0 |
| effect | 30 min after use | Stay: 0 | Stay: 0 | Stay: 0 | Stay: 0 | Stay: 0 |
| sustaining | 60 min after use | Stay: 0 | Stay: 0 | Stay: 0 | Stay: 0 | Stay: 0 |
| time | 90 min after use | Stay: 0 | Stay: 0 | Stay: 0 | Stay: 0 | Stay: 0 |
| | 120 min after use | Stay: 1 | Stay: 0 | Stay: 0 | Stay: 1 | Stay: 0 |
| | 150 min after use | Stay: 1 | Stay: 0 | Stay: 0 | Stay: 1 | Stay: 0 |
| | 180 min after use | | Stay: 0 | Stay: 0 | | Stay: 0 |
| | 210 min after use | | Stay: 2 | Stay: 0 | | Stay: 0 |
| | 240 min after use | | | Stay: 0 | | Stay: 0 |
| | 270 min after use | | | Stay: 0 | | Stay: 2 |
| | 300 min after use | | | Stay: 0 | | |
| | 330 min after use | | | Stay: 1 | | |
| | 360 min after use | | | Stay: 1 | | |
| | 390 min after use | | | | | |
| | 420 min after use | | | | | |
| | 450 min after use | | | | | |
| | 480 nm after use | | | | | |

| | | | | | |
|---|---|---|---|---|---|
| Feeling (dry feeling during applying onto the skin; 5-grade evaluation) | 3.7 | 3.4 | 2.7 | 5.0 | 4.7 |
| Long-term storage stability | B | B | B | B | B |

| | | | | | Volume | | | | |
|---|---|---|---|---|---|---|---|---|---|
| | | | | | median | | | | |
| | | Surface | | Wetting | particle | | | | |
| | | tension at | Viscosity | tension | diameter | | | | |
| | | 25° C. | at 23° C. | at 23° C. | (D50) | Example | | | |
| % by mass | | [mN/m] | [mPa · s] | [mN/m] | [μm] | 48 | 49 | 50 | 51 |
| (A1) | Dimethylpolysiloxane | 19.0 | 5 | — | — | 0.00 | 0.00 | 0.00 | 0.00 |
| (A2) | Dimethylpolysiloxane | 21.0 | 10 | — | — | 0.00 | 0.00 | 0.00 | 0.00 |
| (A3) | Dimethylpolysiloxane | 21.0 | 60 | — | — | 0.00 | 0.00 | 0.00 | 0.00 |
| (A4) | Dimethylpolysiloxane | 21.0 | 210 | — | — | 0.00 | 0.00 | 0.00 | 0.00 |
| (A5) | Isopropyl myristate | 28.0 | 5 | — | — | 45.00 | 0.00 | 0.00 | 0.00 |
| (A6) | Squalane | 30.0 | 42 | — | — | 0.00 | 15.00 | 30.00 | 45.00 |
| (A7) | Cetyl-1,3-dimethyl- | 28.0 | 7 | — | — | 0.00 | 0.00 | 0.00 | 0.00 |
| | phenyl ether | | | | | | | | |
| (A8) | Polypropylene glycol/ | 34.0 | 160 | — | — | 0.00 | 0.00 | 0.00 | 0.00 |
| | 1,3-butylene glycol | | | | | | | | |
| | (mixture having a | | | | | | | | |
| | mass ratio of 3/7) | | | | | | | | |
| (a1) | Dimethylpolysiloxane | 21.0 | 560 | — | — | 0.00 | 0.00 | 0.00 | 0.00 |
| (a2) | Glycerin | 65.0 | 950 | — | — | 0.00 | 0.00 | 0.00 | 0.00 |
| (a3) | Dimethylpolysiloxane | 21.0 | 4500 | — | — | 0.00 | 0.00 | 0.00 | 0.00 |
| (a4) | 1,3-Propanediol | 48.0 | 53 | — | — | 0.00 | 0.00 | 0.00 | 0.00 |
| (B3) | (Lauryl methacrylate/ | — | — | 42 | 3.2 | 5.00 | 5.00 | 5.00 | 5.00 |
| | Na methacrylate) | | | | | | | | |
| | crosspolymer | | | | | | | | |
| (B5) | Polymethyl methacrylate | — | — | 44 | 12.5 | 1.67 | 1.67 | 1.67 | 1.67 |
| (B9) | (Vinyl dimethicone/ | — | — | 38 | 24.9 | 3.33 | 3.33 | 3.33 | 3.33 |
| | methiconesilsesquioxane) | | | | | | | | |
| | crosspolymer | | | | | | | | |

| (D1) | (Ammonium acryloyldimethyltaurate/ | 0.50 | 0.50 | 0.50 | 0.50 |
|---|---|---|---|---|---|
| | beheneth-25 methacrylate) crosspolymer | | | | |
| (C) | Water | 44.50 | 74.50 | 59.50 | 44.50 |
| Total | | 100.0 | 100.0 | 100.0 | 100.0 |
| Total content of particles (% by mass) | | 10.0 | 10.0 | 10.0 | 10.0 |
| volume median particle diameter of particles (D50) [μm] | | 10.2 | 10.2 | 10.2 | 10.2 |
| Content of component (A) (% by mass) | | 45.0 | 15.0 | 30.0 | 45.0 |
| Content of component (B) (% by mass) | | 10.0 | 10.0 | 10.0 | 10.0 |
| Mass ratio (A)/(B) | | 4.5 | 1.5 | 3.0 | 4.5 |
| Mass ratio (A)/(C) | | 1.0 | 0.2 | 0.5 | 1.0 |
| Repellent | Repellent effect immediately after use | Stay: 0 | Stay: 0 | Stay: 0 | Stay: 0 |
| effect | 30 min after use | Stay: 0 | Stay: 0 | Stay: 0 | Stay: 0 |
| sustaining | 60 min after use | Stay: 0 | Stay: 0 | Stay: 0 | Stay: 0 |
| time | 90 min after use | Stay: 0 | Stay: 0 | Stay: 0 | Stay: 0 |
| | 120 min after use | Stay: 0 | Stay: 1 | Stay: 0 | Stay: 0 |
| | 150 min after use | Stay: 0 | Stay: 1 | Stay: 0 | Stay: 0 |
| | 180 min after use | Stay: 0 | | Stay: 0 | Stay: 0 |
| | 210 min after use | Stay: 0 | | Stay: 1 | Stay: 0 |
| | 240 min after use | Stay: 0 | | Stay: 1 | Stay: 0 |
| | 270 min after use | Stay: 0 | | | Stay: 0 |
| | 300 min after use | Stay: 0 | | | Stay: 0 |
| | 330 min after use | Stay: 0 | | | Stay: 1 |
| | 360 min after use | Stay: 0 | | | Stay: 1 |
| | 390 min after use | Stay: 2 | | | |
| | 420 min after use | | | | |
| | 450 min after use | | | | |
| | 480 nm after use | | | | |

| | | | | |
|---|---|---|---|---|
| Feeling (dry feeling during applying onto the skin; 5-grade evaluation) | 4.0 | 5.0 | 4.7 | 4.0 |
| Long-term storage stability | B | B | B | B |

TABLE 7

| | | | | | Volume | | | | |
|---|---|---|---|---|---|---|---|---|---|
| | | | | | median | | | | |
| | | Surface | | Wetting | particle | | | | |
| | | tension | Viscosity | tension | diameter | | | | |
| | | at 25° C. | at 23° C. | at 23° C. | (D50) | Example | | | |
| % by mass | | [mN/m] | [mPa · s] | [mN/m] | [μm] | 52 | 53 | 54 | 55 |
| (A1) | Dimethylpolysiloxane | 19.0 | 5 | — | — | 0.00 | 0.00 | 0.00 | 0.00 |
| (A2) | Dimethylpolysiloxane | 21.0 | 10 | — | — | 0.00 | 0.00 | 0.00 | 0.00 |
| (A3) | Dimethylpolysiloxane | 21.0 | 60 | — | — | 0.00 | 0.00 | 0.00 | 0.00 |
| (A4) | Dimethylpolysiloxane | 21.0 | 210 | — | — | 0.00 | 0.00 | 0.00 | 0.00 |
| (A5) | Isopropyl myristate | 28.0 | 5 | — | — | 0.00 | 0.00 | 0.00 | 0.00 |
| (A6) | Squalane | 30.0 | 42 | — | — | 0.00 | 0.00 | 0.00 | 0.00 |
| (A7) | Cetyl-1,3-dimethylbutyl ether | 28.0 | 7 | — | — | 15.00 | 30.00 | 45.00 | 0.00 |
| (A8) | Polypropylene glycol/ | 34.0 | 160 | — | — | 0.00 | 0.00 | 0.00 | 15.00 |
| | 1,3-butylene glycol (mixture | | | | | | | | |
| | having a mass ratio of 3/7) | | | | | | | | |
| (a1) | Dimethylpolysiloxane | 21.0 | 560 | — | — | 0.00 | 0.00 | 0.00 | 0.00 |
| (a2) | Glycerin | 65.0 | 950 | — | — | 0.00 | 0.00 | 0.00 | 0.00 |
| (a3) | Dimethylpolysiloxane | 21.0 | 4500 | — | — | 0.00 | 0.00 | 0.00 | 0.00 |
| (a4) | 1,3-Propanediol | 48.0 | 53 | — | — | 0.00 | 0.00 | 0.00 | 0.00 |
| (B3) | (Lauryl methacrylate/Na | — | — | 42 | 3.2 | 5.00 | 5.00 | 5.00 | 5.00 |
| | methacrylate) crosspolvmer | | | | | | | | |
| (B5) | Polymethyl methacrylate | — | — | 44 | 12.5 | 1.67 | 1.67 | 1.67 | 1.67 |
| (B9) | (Vinyl dimethicone/ | — | — | 38 | 24.9 | 3.33 | 3.33 | 3.33 | 3.33 |
| | methiconesilsesquioxane) | | | | | | | | |
| | crosspolymer | | | | | | | | |
| (D1) | (Ammonium acryloyldimethyltaurate/ | | | | | 0.50 | 0.50 | 0.50 | 0.50 |
| | beheneth-25 methacrylate) crosspolymer | | | | | | | | |
| (C) | Water | | | | | 74.50 | 59.50 | 44.50 | 74.50 |
| Total | | | | | | 100.0 | 100.0 | 100.0 | 100.0 |
| Total content of particles (% by mass) | | | | | | 10.0 | 10.0 | 10.0 | 10.0 |
| volume median particle diameter of particles (D50) [μm] | | | | | | 10.2 | 10.2 | 10.2 | 10.2 |
| Content of component (A) (% by mass) | | | | | | 15.0 | 30.0 | 45.0 | 15.0 |
| Content of component (B) (% by mass) | | | | | | 10.0 | 10.0 | 10.0 | 10.0 |
| Mass ratio (A)/(B) | | | | | | 1.5 | 3.0 | 4.5 | 1.5 |
| Mass ratio (A)/(C) | | | | | | 0.2 | 0.5 | 1.0 | 0.2 |
| Repellent | Repellent effect immediately after use | | | | | Stay: 0 | Stay: 0 | Stay: 0 | Stay: 0 |
| effect | 30 min after use | | | | | Stay: 0 | Stay: 0 | Stay: 0 | Stay: 0 |
| sustaining | 60 min after use | | | | | Stay: 0 | Stay: 0 | Stay: 0 | Stay: 0 |
| time | 90 min after use | | | | | Stay: 0 | Stay: 0 | Stay: 0 | Stay: 0 |
| | 120 min after use | | | | | Stay: 1 | Stay: 0 | Stay: 0 | Stay: 1 |
| | 150 min after use | | | | | Stay: 1 | Stay: 0 | Stay: 0 | Stay: 1 |
| | 180 min after use | | | | | | Stay: 0 | Stay: 0 | |
| | 210 min after use | | | | | | Stay: 2 | Stay: 0 | |
| | 240 min after use | | | | | | | Stay: 0 | |
| | 270 min after use | | | | | | | Stay: 0 | |
| | 300 min after use | | | | | | | Stay: 0 | |
| | 330 min after use | | | | | | | Stay: 1 | |
| | 360 min after use | | | | | | | Stay: 1 | |
| | 390 min after use | | | | | | | | |
| | 420 min after use | | | | | | | | |
| | 450 min after use | | | | | | | | |
| | 480 nm after use | | | | | | | | |
| | 510 min after use | | | | | | | | |
| | 540 min after use | | | | | | | | |
| | 570 min after use | | | | | | | | |
| | 600 min after use | | | | | | | | |
| | 630 min after use | | | | | | | | |
| Feeling (dry feeling during applying onto the skin; | | | | | | 4.5 | 4.2 | 3.5 | 3.0 |
| 5-grade evaluation) | | | | | | | | | |
| Long-term storage stability | | | | | | B | B | B | A |
| | | | | | Volume | | | | |
| | | | | | median | | | | |
| | | Surface | | Wetting | particle | | | | |
| | | tension | Viscosity | tension | diameter | | | Comparative | |
| | | at 25° C. | at 23° C. | at 23° C. | (D50) | Example | | Example | |
| % by mass | | [mN/m] | [mPa · s] | [mN/m] | [μm] | 56 | 57 | 7 | 8 |
| (A1) | Dimethylpolysiloxane | 19.0 | 5 | — | — | 0.00 | 0.00 | 0.00 | 0.00 |
| (A2) | Dimethylpolysiloxane | 21.0 | 10 | — | — | 0.00 | 0.00 | 0.00 | 0.00 |
| (A3) | Dimethylpolysiloxane | 21.0 | 60 | — | — | 0.00 | 0.00 | 0.00 | 0.00 |
| (A4) | Dimethylpolysiloxane | 21.0 | 210 | — | — | 0.00 | 0.00 | 0.00 | 0.00 |
| (A5) | Isopropyl myristate | 28.0 | 5 | — | — | 0.00 | 0.00 | 0.00 | 0.00 |
| (A6) | Squalane | 30.0 | 42 | — | — | 0.00 | 0.00 | 0.00 | 0.00 |
| (A7) | Cetyl-1,3-dimethylbutyl ether | 28.0 | 7 | — | — | 0.00 | 0.00 | 0.00 | 0.00 |
| (A8) | Polypropvlene glycol/ | 34.0 | 160 | — | — | 30.00 | 45.00 | 0.00 | 0.00 |
| | 1,3-butylene glycol (mixture | | | | | | | | |
| | having a mass ratio of 3/7) | | | | | | | | |
| (a1) | Dimethylpolysiloxane | 21.0 | 560 | — | — | 0.00 | 0.00 | 30.00 | 0.00 |
| (a2) | Glycerin | 65.0 | 950 | — | — | 0.00 | 0.00 | 0.00 | 30.00 |
| (a3) | Dimethylpolysiloxane | 21.0 | 4500 | — | — | 0.00 | 0.00 | 0.00 | 0.00 |
| (a4) | 1,3-Propanediol | 48.0 | 53 | — | — | 0.00 | 0.00 | 0.00 | 0.00 |
| (B3) | (Lauryl methacrylate/Na | — | — | 42 | 3.2 | 5.00 | 5.00 | 5.00 | 5.00 |
| | methacrylate) crosspolvmer | | | | | | | | |
| (B5) | Polymethyl methacrylate | — | — | 44 | 12.5 | 1.67 | 1.67 | 1.67 | 1.67 |
| (B9) | (Vinyl dimethicone/ | — | — | 38 | 24.9 | 3.33 | 3.33 | 3.33 | 3.33 |
| | methiconesilsesquioxane) | | | | | | | | |
| | crosspolymer | | | | | | | | |
| (D1) | (Ammonium acryloyldimethyltaurate/ | | | | | 0.50 | 0.50 | 0.50 | 0.50 |
| | beheneth-25 methacrylate) crosspolymer | | | | | | | | |
| (C) | Water | | | | | 59.50 | 44.50 | 59.50 | 59.50 |
| Total | | | | | | 100.0 | 100.0 | 100.0 | 100.0 |
| Total content of particles (% by mass) | | | | | | 10.0 | 10.0 | 10.0 | 10.0 |
| volume median particle diameter of particles (D50) [μm] | | | | | | 10.2 | 10.2 | 10.2 | 10.2 |
| Content of component (A) (% by mass) | | | | | | 30.0 | 45.0 | 0.0 | 0.0 |
| Content of component (B) (% by mass) | | | | | | 10.0 | 10.0 | 10.0 | 10.0 |
| Mass ratio (A)/(B) | | | | | | 3.0 | 4.5 | 0.0 | 0.0 |
| Mass ratio (A)/(C) | | | | | | 0.5 | 1.0 | 0.0 | 0.0 |
| Repellent | Repellent effect immediately after use | | | | | Stay: 0 | Stay: 0 | Stay: 0 | Stay: 2 |
| effect | 30 min after use | | | | | Stay: 0 | Stay: 0 | Stay: 0 | |
| sustaining | 60 min after use | | | | | Stay: 0 | Stay: 0 | Stay: 0 | |
| time | 90 min after use | | | | | Stay: 0 | Stay: 0 | Stay: 2 | |
| | 120 min after use | | | | | Stay: 0 | Stay: 0 | | |
| | 150 min after use | | | | | Stay: 0 | Stay: 0 | | |
| | 180 min after use | | | | | Stay: 0 | Stay: 0 | | |
| | 210 min after use | | | | | Stay: 2 | Stay: 0 | | |
| | 240 min after use | | | | | | Stay: 0 | | |
| | 270 min after use | | | | | | Stay: 0 | | |
| | 300 min after use | | | | | | Stay: 2 | | |
| | 330 min after use | | | | | | | | |
| | 360 min after use | | | | | | | | |
| | 390 min after use | | | | | | | | |
| | 420 min after use | | | | | | | | |
| | 450 min after use | | | | | | | | |
| | 480 nm after use | | | | | | | | |
| | 510 min after use | | | | | | | | |
| | 540 min after use | | | | | | | | |
| | 570 min after use | | | | | | | | |
| | 600 min after use | | | | | | | | |
| | 630 min after use | | | | | | | | |
| Feeling (dry feeling during applying onto the skin; | | | | | | 2.6 | 2.0 | 1.0 | 1.0 |
| 5-grade evaluation) | | | | | | | | | |
| Long-term storage stability | | | | | | A | A | A | A |
| | | | | | Volume | | | | |
| | | | | | median | | | | |
| | | Surface | | Wetting | particle | | | | |
| | | tension | Viscosity | tension | diameter | | | | |
| | | at 25° C. | at 23° C. | at 23° C. | (D50) | Comparative Example | | | |
| % by mass | | [mN/m] | [mPa · s] | [mN/m] | [μm] | 9 | 10 | 11 | 12 |
| (A1) | Dimethylpolysiloxane | 19.0 | 5 | — | — | 0.00 | 0.00 | 10.00 | 60.00 |
| (A2) | Dimethylpolysiloxane | 21.0 | 10 | — | — | 0.00 | 0.00 | 0.00 | 0.00 |
| (A3) | Dimethylpolysiloxane | 21.0 | 60 | — | — | 0.00 | 0.00 | 0.00 | 0.00 |
| (A4) | Dimethylpolysiloxane | 21.0 | 210 | — | — | 0.00 | 0.00 | 0.00 | 0.00 |
| (A5) | Isopropyl myristate | 28.0 | 5 | — | — | 0.00 | 0.00 | 0.00 | 0.00 |
| (A6) | Squalane | 30.0 | 42 | — | — | 0.00 | 0.00 | 0.00 | 0.00 |
| (A7) | Cetyl-1,3-dimethylbutyl ether | 28.0 | 7 | — | — | 0.00 | 0.00 | 0.00 | 0.00 |
| (A8) | Polypropvlene glycol/ | 34.0 | 160 | — | — | 0.00 | 0.00 | 0.00 | 0.00 |
| | 1,3-butylene glycol (mixture | | | | | | | | |
| | having a mass ratio of 3/7) | | | | | | | | |
| (a1) | Dimethylpolysiloxane | 21.0 | 560 | — | — | 0.00 | 0.00 | 0.00 | 0.00 |
| (a2) | Glycerin | 65.0 | 950 | — | — | 0.00 | 0.00 | 0.00 | 0.00 |
| (a3) | Dimethylpolysiloxane | 21.0 | 4500 | — | — | 30.00 | 0.00 | 0.00 | 0.00 |
| (a4) | 1,3-Propanediol | 48.0 | 53 | — | — | 0.00 | 30.00 | 0.00 | 0.00 |
| (B3) | (Lauryl methacrylate/Na | — | — | 42 | 3.2 | 5.00 | 5.00 | 5.00 | 5.00 |
| | methacrylate) crosspolvmer | | | | | | | | |
| (B5) | Polymethyl methacrylate | — | — | 44 | 12.5 | 1.67 | 1.67 | 1.67 | 1.67 |
| (B9) | (Vinyl dimethicone/ | — | — | 38 | 24.9 | 3.33 | 3.33 | 3.33 | 3.33 |
| | methiconesilsesquioxane) | | | | | | | | |
| | crosspolymer | | | | | | | | |

| (D1) | (Ammonium acryloyldimethyltaurate/ | 0.50 | 0.50 | 0.50 | 0.50 |
|---|---|---|---|---|---|
| | beheneth-25 methacrylate) crosspolymer | | | | |
| (C) | Water | 59.50 | 59.50 | 79.50 | 29.50 |
| Total | | 100.0 | 100.0 | 100.0 | 100.0 |
| Total content of particles (% by mass) | | 10.0 | 10.0 | 10.0 | 10.0 |
| volume median particle diameter of particles (D50) [μm] | | 10.2 | 10.2 | 10.2 | 10.2 |
| Content of component (A) (% by mass) | | 0.0 | 0.0 | 10.0 | 60.0 |
| Content of component (B) (% by mass) | | 10.0 | 10.0 | 10.0 | 10.0 |
| Mass ratio (A)/(B) | | 0.0 | 0.0 | 1.0 | 6.0 |
| Mass ratio (A)/(C) | | 0.0 | 0.0 | 0.1 | 2.0 |
| Repellent | Repellent effect immediately after use | Stay: 0 | Stay: 0 | Stay: 0 | Stay: 0 |
| effect | 30 min after use | Stay: 0 | Stay: 0 | Stay: 0 | Stay: 0 |
| sustaining | 60 min after use | Stay: 2 | Stay: 2 | Stay: 2 | Stay: 0 |
| time | 90 min after use | | | | Stay: 0 |
| | 120 min after use | | | | Stay: 0 |
| | 150 min after use | | | | Stay: 0 |
| | 180 min after use | | | | Stay: 0 |
| | 210 min after use | | | | Stay: 0 |
| | 240 min after use | | | | Stay: 0 |
| | 270 min after use | | | | Stay: 0 |
| | 300 min after use | | | | Stay: 0 |
| | 330 min after use | | | | Stay: 0 |
| | 360 min after use | | | | Stay: 0 |
| | 390 min after use | | | | Stay: 0 |
| | 420 min after use | | | | Stay: 0 |
| | 450 min after use | | | | Stay: 0 |
| | 480 nm after use | | | | Stay: 0 |
| | 510 min after use | | | | Stay: 0 |
| | 540 min after use | | | | Stay: 0 |
| | 570 min after use | | | | Stay: 0 |
| | 600 min after use | | | | Stay: 0 |
| | 630 min after use | | | | Stay: 2 |

| | | | | |
|---|---|---|---|---|
| Feeling (dry feeling during applying onto the skin; | 1.0 | 1.3 | 5.0 | 3.3 |
| 5-grade evaluation) | | | | |
| Long-term storage stability | C | A | B | C |

From Tables 2 to 4 and Tables 6 to 7, it is noted that the compositions of the present invention each containing predetermined amounts of the components (A) to (C) are able to keep the repellent sustaining effect over a long period of time of 2 hours or more, have a good dry feeling on the occasion of applying onto the skin, and are excellent in the long-term storage stability.

### INDUSTRIAL APPLICABILITY

In accordance with the present invention, it is possible to provide an oil-in-water type pest repellent composition and an oil-in-water type pest stay inhibition composition, each of which is excellent in the pest repellent sustaining effect against pests, particularly flying pests, is safe, is good in the feeling on the occasion of applying onto the skin, and is excellent in the long-term storage stability, a method for repelling pests, and a method for inhibiting pest stay.

What is claimed is:

1. An oil-in-water type pest repellent composition comprising the following components (A) to (C):
(A) 13% by mass or more and 50% by mass or less of at least one non-volatile liquid oily component selected from the group consisting of a silicone oil, an ester oil, an ether oil, a hydrocarbon oil, an aliphatic alcohol, and a polyhydric alcohol, the non-volatile liquid oily component having a surface tension at 25° C. of 40 mN/m or less and a viscosity at 23° C. as measured with a B-type rotational viscometer of 400 mPa·s or less;
(B) 0.5% by mass or more and 35% by mass or less of hydrophobic particles having a volume median particle diameter of 0.1 μm or more and 40 μm or less; and
(C) 30% by mass or more and 86.5% by mass or less of water.

2. An oil-in-water type pest stay inhibition composition comprising the following components (A) to (C):
(A) 13% by mass or more and 50% by mass or less of at least one non-volatile liquid oily component selected from the group consisting of a silicone oil, an ester oil, an ether oil, a hydrocarbon oil, an aliphatic alcohol, and a polyhydric alcohol, the non-volatile liquid oily component having a surface tension at 25° C. of 40 mN/m or less and a viscosity at 23° C. as measured with a B-type rotational viscometer of 400 mPa·s or less;
(B) 0.5% by mass or more and 35% by mass or less of hydrophobic particles having a volume median particle diameter of 0.1 μm or more and 40 μm or less; and
(C) 30% by mass or more and 86.5% by mass or less of water.

3. The composition according to claim 1, wherein the component (A) is at least one selected from the group consisting of a silicone oil, an ester oil, and a hydrocarbon oil.

4. The composition according to claim 1, wherein the component (B) contains resin particles constituted of at least one resin selected from the group consisting of a silicone resin, an acrylic resin, and a polyurethane resin.

5. A method for repelling pests, comprising applying the composition according to claim 1 onto the skin surface of a human.

6. A method for inhibiting pest stay, comprising attaching the composition according to claim 2 onto limbs of a pest to prevent the pest from staying on the skin of a human.

7. The composition according to claim 1, wherein the component (B) comprises hydrophobic organic particles.

8. The composition according to claim 1, wherein the content of an existing pest repellent than the component (A) is 15% by mass or less.

9. The composition according to claim 1, wherein the content of p-menthane-3,8-diol is 0.5% by mass or less.

10. The composition according to claim 1, wherein the surface tension at 25° C. of the component (A) is 15 mN/m or more.

11. The composition according to claim 1, wherein the viscosity at 23° C. of the component (A) as measured with a B-type rotational viscometer is 1 mPa·s or more.

12. The composition according to claim 1, wherein the mass ratio [(A)/(B)] of the component (A) to the component (B) is 1 or more and 50 or less.

13. The composition according to claim 1, wherein the mass ratio [(A)/(C)] of the component (A) to the component (C) is 0.15 or more and 1.5 or less.

14. The composition according to claim 1, wherein in a distribution curve of particle diameters of the component (B) as measured using a laser scattering particle size distribution analyzer, a volume proportion of particle diameters of 45 μm or more relative to the volume of the whole of particles is 15% or less.

15. The composition according to claim 1, wherein the component (B) does not comprise titanium oxide in which the surface thereof is treated by a dimethylpolysiloxane.

16. The composition according to claim 1, further comprises, as a component (D), a thickener in an amount of 0.01% by mass or more and 5% by mass or less.

17. The composition according to claim 1, wherein the content of a surfactant in the composition is 0.01% by mass or more and 3% by mass or less.
